(19) United States
(12) Patent Application Publication
(10) Pub. No.: US 2026/0294748 A1
(43) Pub. Date: Oct. 1, 2026

(54) COMPOSITIONS AND METHODS FOR SKIN PROTECTION AND HYDRATION

(71) Applicant: TRUSKIN, LLC, Seattle, WA (US)

(72) Inventor: Jalshree Trivedi, Newark, CA (US)

(73) Assignee: TRUSKIN, LLC, Seattle, WA (US)

(21) Appl. No.: 19/633,838

(22) Filed: Mar. 30, 2026

Related U.S. Application Data
(60) Provisional application No. 63/781,276, filed on Mar. 31, 2025.

(51) Int. Cl. A61K 8/02; A61K 8/27; A61K 8/67; A61K 8/9789; A61Q 17/04; A61Q 19/00

(52) U.S. Cl. CPC A61K 8/0216; A61K 8/27; A61K 8/676; A61K 8/9789; A61Q 17/04; A61Q 19/005; A61K 2800/43

(57) ABSTRACT

Provided herein are compositions for use as topical formulations for sun protection and/or skin sensitivity. The disclosed compositions comprise essentially natural ingredients, one or more of which help manage, reduce and/or ameliorate symptoms of discomfort associated with UV exposure and/or skin sensitivity.

### CROSS REFERENCE TO RELATED APPLICATION

This application claims priority to U.S. Provisional Patent Application Ser. No. 63/781,276, filed Mar. 31, 2025, and titled “COMPOSITIONS AND METHODS FOR SKIN PROTECTION AND HYDRATION,” which is incorporated by reference herein in its entirety.

### BACKGROUND

### 1. Field

The present disclosure broadly relates to compositions for use as skin protectants and sunscreen.

### 2. Discussion of Related Art

The harmful effect of sun exposure on the skin is well documented. Leaving skin unprotected invites harmful UV rays, causing sunburns, premature aging, heightened skin cancer risk, uneven skin tone and eye damage. Most of the damage associated with sun exposure is attributed to UV (particularly UVA and UVB) rays, but infrared (IR) rays have also been shown to induce a significant production of free radicals in dermis and diminish skin's antioxidant capacity. There is a need for improved sunscreen products. However, a large portion of the population (e.g., almost 60% of women and 40% of men) suffer from sensitive skin, characterized by erythema (skin reddening), prickling, burning and discomfort generally caused by an overreaction to non-pathogenic stimulus such as wind, heat, cold, water, or stress. Designing sunscreens for these populations are difficult as many commercially available sunscreens contain skin irritants. Therefore, there is a need for improved sun protection compositions adapted and suited for sensitive skin populations.

### SUMMARY

In various aspects, the present disclosure is directed to a composition comprising zinc oxide, an antioxidant, and at least one plant extract.

In various aspects, the antioxidant comprises ascorbic acid or a derivative thereof. In some aspects, the antioxidant comprises sodium ascorbyl phosphate (SAP). In various aspects, the antioxidant can comprise up to 1% by weight of the total composition.

In various aspects, the composition comprises at least one plant extract selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In various aspects, the composition comprises at least two plant extracts selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In various aspects, the composition comprises at least three plant extracts selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In various aspects, the composition comprises Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

In various aspects, the composition comprises up to 1% of Camellia sinensis (green tea) leaf extract, up to 1% of Opuntia ficus-indica stem (prickly pear) extract, up to 1% of Caesalpinia spinosa (spiny holdback) fruit pod extract, and/or up to 1% of Helianthus annuus (sunflower) sprout extract. In various aspects, the composition comprises up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract.

In various aspects, the composition may comprise about 10% to about 30% zinc oxide. For example, in some aspects, the composition may comprise about 12% to about 18% zinc oxide. In some aspects, the composition comprises about 16.1% zinc oxide.

In various aspects, the composition comprises 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract.

In any of the foregoing or related aspects, the compositions disclosed herein may further comprise at least one skin conditioner. In some aspects, the at least one skin conditioner comprises caprylic/capric triglyceride, coconut alkanes, isopropyl isostearate, or any combination thereof. In some aspects, the composition comprises at least one, at least two, or at least three skin conditioners selected from caprylic/capric triglyceride, coconut alkanes, and isopropyl isostearate. In various aspects, the composition comprises caprylic/capric triglyceride, coconut alkanes and isopropyl isostearate. In various aspects, the at least one skin conditioner comprises about 15% to about 35% by weight of the total composition.

In some aspects, the composition comprises a first skin conditioner comprising about 15% to about 25% by weight of the total composition, a second skin conditioner comprising about 1% to 5% by weight of the total composition, and a third skin conditioner comprising about 1% to 5% by weight of the total composition. For example, in some aspects, the composition may comprise 15% to about 25% caprylic/capric triglyceride, about 1% to 5% isopropyl isostearate, and about 1% to 5% coconut alkanes.

In further aspects, the compositions disclosed herein may further comprise a humectant, a nonaqueous solvent, and/or a surfactant, emulsifier, emulsion stabilizer, or any combination thereof.

For example, in some aspects, the compositions disclosed herein comprise a humectant. In some aspects, the humectant is at a concentration of about 1 to 5% of the total composition. In some aspects, the humectant comprises glycerin.

In various aspects, the compositions disclosed herein comprise a nonaqueous solvent. In some aspects, the nonaqueous solvent is at a concentration of about 1 to 5% of the total composition. In some aspects, the non-aqueous solvent comprises C13-C15 alkane.

In various aspects, the compositions disclosed herein may further comprise a surfactant, emulsifier, emulsion stabilizer, or any combination thereof. In various aspects, the surfactant, emulsifier, emulsion stabilizer, or any combination thereof is at a concentration of about 1 to 5% by weight of the total composition. In various aspects, the surfactant, emulsifier, emulsion stabilizer, or any combination thereof comprises polyglyceryl-4 diisostearate/polyhydroxystearate/sebacate.

Accordingly, in various aspects, the compositions disclosed herein may comprise glycerin, C12-C15 alkane and polyglyceryl-4 diisostearate/polyhydroxystearate/sebacate. In some aspects, the compositions disclosed herein comprise 1 to 5% glycerin, 1 to 5% of C13-C15 alkane and 1 to 5% of polyglyceryl-4 diisostearate/polyhydroxystearate/sebacate.

In accord with the foregoing, the compositions disclosed herein may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract, 15% to about 25% caprylic/capric triglyceride, 1% to 5% isopropyl isostearate, 1% to 5% coconut alkanes, 1 to 5% glycerin, 1 to 5% of C13-C15 alkane and 1 to 5% of polyglyceryl-4 diisostearate/polyhydroxystearate/sebacate.

In any of the foregoing or related aspects, the compositions disclosed herein may further comprise one or more additional ingredients. In various aspects, the one or more additional ingredients comprise another skin conditioner, another antioxidant, another humectant, another nonaqueous solvent, an emollient, an emulsifier, a surfactant, a bulking agent, a viscosity controller, a cleanser, a gelling agent, a film former, a preservative, a buffer, or any combination thereof. In various aspects, the one or more additional ingredients are selected from sodium chloride, niacinamide, sodium hyaluronate, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, citric acid, phytosphingosine, cholesterol, sodium lauroyl lactylate, and carbomer. In various aspects, each of the one or more additional ingredients are present at a concentration of up to 1% by weight of the total composition.

In various aspects, the compositions disclosed herein may further comprise one or more colorants. The colorants may comprise, in some aspects, iron oxides.

In further aspects, also provided is a composition comprising zinc oxide, caprylic/capric triglyceride, isopropyl isostearate, coconut alkanes, polyglyceryl-4 diisostearate/polyhydroxystearate/sebacate, C13-15 Alkane, glycerin, sodium ascorbyl phosphate, and at least one plant extract selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

In various aspects, the composition comprises 16.1% zinc oxide, 15 to 25% caprylic/capric triglyceride, 1 to 5% of isopropyl isostearate, 1 to 5% of coconut alkanes, 1 to 5% of polyglyceryl-4 diisostearate/polyhydroxystearate/sebacate, 1 to 5% of C13-15 Alkane, 1 to 5% of glycerin, up to 1% sodium ascorbyl phosphate, up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract. In various aspects, the composition comprises Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

In various aspects, the disclosed compositions may further comprise one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more of sodium chloride, niacinamide, sodium hyaluronate, coco-caprylate/caprate, polyhydroxystearic acid, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, and citric acid, each at a concentration up to 1% by weight of the total composition. In various aspects, the composition comprises sodium chloride, niacinamide, sodium hyaluronate, coco-caprylate/caprate, polyhydroxystearic acid, polyglyceryl-3 polyricinoleate, isostearic acid, lecithin, ceramide NP, ceramide AP, ceramide EOP, sodium ascorbyl phosphate, xanthan gum, maltodextrin, ethylhexylglycerin, caprylyl glycol, hexylene glycol, phenoxyethanol, sodium benzoate, potassium sorbate, and citric acid.

In various aspects, any of the disclosed compositions may be formulated for topical administration. In some aspects, the compositions comprise a topical serum, ointment, or cream. In some aspects, the disclosed compositions comprise a topical serum.

Also provided are methods of protecting a skin surface from sun exposure in a subject in need thereof, the method comprising topically applying a composition disclosed herein to the skin surface. In various aspects, the composition may be topically applied to the skin surface up to 1 hour, up to 2 hours, or up to 3 hours before exposure of the skin surface to a UV source. In any of these or related aspects, the composition applied to the skin surface may have a static sun protection factor (SPF) of between 30 and 40.

In various aspects, protecting a skin surface from sun exposure comprises preventing or ameliorating one or more of the following effects associated with sun exposure: sunburn, sun rash, itching, pain, blistering, allodynia (hypersensitivity), skin redness (erythema), or any other form of discomfort.

Also provided are methods of ameliorating or addressing skin sensitivity in a subject in need thereof, the method comprising topically applying a composition disclosed herein to a skin surface of a subject. In various aspects, ameliorating skin sensitivity comprises reducing irritation, itching, dryness, allodynia (hypersensitivity), erythema (skin redness), or any combination thereof.

In any of the foregoing or related aspects, the subject may be a human.

Also provided is a use of any composition disclosed herein in a method of protecting a skin surface from sun exposure. Also provided is a use of any composition disclosed herein in the preparation of a medicament for protecting a skin surface from sun exposure. Also provided is a use of any composition disclosed herein in a method of ameliorating or addressing skin sensitivity. Also provided is a use of any composition disclosed herein in the preparation of a medicament for of ameliorating or addressing skin sensitivity. In any of these uses or related aspects, the compositions may be formulated for topical administration.

### DETAILED DESCRIPTION

In some aspects, the present disclosure is based on the discovery of improved skin serums that effectively block UV and IR light and improve skin texture, smoothness and comfort. The compositions disclosed herein encompass a unique combination of ingredients that are not only compatible within one another but work in a synergistic manner in improving sun protection and/or skin health. In various aspects, the disclosed compositions are characterized by one or more sunscreen or UV blocking compounds (e.g., zinc oxide), one or more antioxidants (e.g., sodium ascorbyl phosphate (SAP)), and one or more plant extracts (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract). Methods of using these compositions to protect against sun exposure and/or ameliorate one or more symptoms of sensitive skin are also provided.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including,” and “having” are used interchangeably in this disclosure. The terms “comprising,” “including,” and “having” mean to include, but not necessarily be limited to the things so described.

The terms “or” and “and/or” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); Chambers Dictionary of Science and Technology (Walker ed., 1999); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, 9%, 8%, 7%, 6%, 5%, 4%, 3%, 2%, or ±1%.

As used herein, “protect/protecting,” “address/addressing,” or the similar refer to intervention made in response to a physiological condition manifested by a subject or to which a subject may be susceptible. The aim of protecting or addressing includes the alleviation or prevention of symptoms, slowing or stopping the progression or worsening of a condition and/or the remission of the condition.

As used herein, “prevent” or “prevention” refers to eliminating or delaying the onset of a particular disease, disorder or physiological condition (e.g., sunburn), or to the reduction of the degree of severity of a particular disease, disorder or physiological condition (e.g., sunburn), relative to the time and/or degree of onset or severity in the absence of intervention.

As used herein, “individual,” “subject,” and “host,” can be used interchangeably herein and refer to any mammalian subject for whom diagnosis prophylaxis, prevention, or protection is desired, for example, humans, pets, livestock, horses or other animals. As used herein, the term “subject” refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human.

### II. Compositions

Various aspects of the present disclosure are directed to new skin protectant compositions.

In various aspects, the compositions disclosed herein comprise zinc oxide, an antioxidant, and at least one plant extract. In various aspects, the compositions disclosed herein comprise zinc oxide, an antioxidant, at least one skin conditioner and at least one plant extract. In some aspects, the compositions disclosed herein comprise zinc oxide, an antioxidant, at least one plant extract, and at least one humectant. In various aspects, the compositions disclosed herein comprise zinc oxide, an antioxidant, at least one plant extract, and at least one nonaqueous solvent. In some aspects, the compositions disclosed herein comprise zinc oxide, an antioxidant, at least one plant extract and at least one surfactant, emulsifier, or emulsion stabilizer. In various aspects, the compositions disclosed herein comprise zinc oxide, an antioxidant, at least one plant extract, at least one skin conditioner, and at least one humectant. In various aspects, the compositions disclosed herein comprise zinc oxide, an antioxidant, at least one plant extract, at least one skin conditioner, and at least one nonaqueous solvent. In various aspects, the compositions disclosed herein comprise zinc oxide, an antioxidant, at least one plant extract, at least one skin conditioner, and at least one surfactant, emulsifier, or emulsion stabilizer. In various aspects, the compositions disclosed herein comprise zinc oxide, an antioxidant, at least one plant extract, at least one humectant, and at least one surfactant, emulsifier, or emulsion stabilizer. In various aspects, the compositions disclosed herein comprise zinc oxide, an antioxidant, at least one plant extract, and at least one nonaqueous solvent, and at least one surfactant, emulsifier, or emulsion stabilizer. In various aspects, the compositions disclosed herein comprise zinc oxide, an antioxidant, at least one plant extract, at least one humectant, and at least one nonaqueous solvent. In various aspects, the compositions disclosed herein comprise zinc oxide, an antioxidant, at least one plant extract, at least one skin conditioner, at least one humectant, and at least one nonaqueous solvent. In various aspects, the compositions disclosed herein comprise zinc oxide, an antioxidant, at least one plant extract, at least one skin conditioner, at least one humectant, and at least one surfactant, emulsifier, or emulsion stabilizer. In various aspects, the compositions disclosed herein comprise zinc oxide, an antioxidant, at least one plant extract, at least one skin conditioner, at least one surfactant, emulsifier, or emulsion stabilize, and at least one nonaqueous solvent. In various aspects, the compositions disclosed herein comprise zinc oxide, an antioxidant, at least one plant extract, at least one humectant, at least one surfactant, emulsifier, or emulsion stabilize, and at least one nonaqueous solvent. In some aspects, the compositions disclosed herein comprise zinc oxide, an antioxidant, at least one plant extract, at least one skin conditioner, at least one humectant, at least one nonaqueous solvent, and at least one surfactant, emulsifier, or emulsion stabilizer.

Exemplary components that may be included in these compositions are disclosed herein below.

### A. Ingredients

### 1. Zinc Oxide

Zinc oxide is used to absorb ultraviolet light. It is the broadest spectrum UVA and UVB absorber that is approved for sunscreen and is photostable. In some aspects, the compositions disclosed herein comprise about 10 to 30% by weight of zinc oxide. For example, in some aspects, the composition comprises at least, at most, exactly, or about 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 30% zinc oxide. In some aspects, the compositions comprise about 12 to 18% zinc oxide (e.g., at least, at most, exactly, or about 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18% zinc oxide). In some aspects the composition comprises at least at most, exactly, or about 16.1% zinc oxide.

### 2. Antioxidant

Antioxidants are molecules that inhibit oxidation (usually occurring as autoxidation), a chemical reaction that can produce free radicals. In various aspects, the antioxidant comprises ascorbic acid (Vitamin C) or a derivative thereof. In some aspects, the derivative of ascorbic acid is sodium ascorbyl phosphate (SAP). Accordingly, in various aspects, the compositions disclosed herein comprise sodium ascorbyl phosphate (SAP). In various aspects, the compositions disclosed herein comprise about 0.001% to 5% of SAP. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% of SAP. In some aspects, the composition comprises up to 1.0% of SAP. In some aspects, the composition comprises at least, at most, exactly, or about 0.1% SAP.

### 3. Plant Extracts

In various aspects, the composition comprises at least one plant extract. In various aspects, the plant extract is selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In some aspects, the composition comprises at least one plant extract selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In some aspects, the composition comprises at least two plant extracts selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In some aspects, the composition comprises at least three plant extracts selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In some aspects, the composition comprises Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

Camellia sinensis (Green Tea) Leaf Extract

In various aspects, the composition comprises Camellia sinensis (green tea) leaf extract. In some aspects, the compositions comprise about 0.001% to 5% of Camellia sinensis (green tea) leaf extract. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% of Camellia sinensis (green tea) leaf extract. In some aspects, the composition comprises up to 1.0% of Camellia sinensis (green tea) leaf extract.

Opuntia ficus-Indica Stem (Prickly Pear) Extract

In various aspects, the composition comprises Opuntia ficus-indica stem (prickly pear) extract. In some aspects, the compositions comprise about 0.001% to 5% of Opuntia ficus-indica stem (prickly pear) extract. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% of Opuntia ficus-indica stem (prickly pear) extract. In some aspects, the composition comprises up to 1.0% of Opuntia ficus-indica stem (prickly pear) extract.

In some aspects, the Opuntia ficus-indica stem (prickly pear) extract is provided as a part of a prepared formulation (e.g., AQUACACTEEN®) which contains Opuntia ficus-indica stem extract along with other ingredients (glycerin and phenoxyethanol) in an aqueous solvent. In some aspects, the AQUACACTEEN® is incorporated into the composition at a concentration of about 0.5% to 2%. Accordingly, in some aspects, the composition may comprise at least, at most, exactly, or about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0% of AQUACACTEEN. In some aspects, the composition comprises 0.5% AQUACACTEEN.

Caesalpinia spinosa (Spiny Holdback) Fruit Pod Extract

In various aspects, the composition comprises Caesalpinia spinosa (spiny holdback) fruit pod extract. In some aspects, the compositions comprise about 0.001% to 5% of Caesalpinia spinosa (spiny holdback) fruit pod extract. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% of Caesalpinia spinosa (spiny holdback) fruit pod extract. In some aspects, the composition comprises up to 1.0% of Caesalpinia spinosa (spiny holdback) fruit pod extract.

In some aspects, the Caesalpinia spinosa (spiny holdback) fruit pod extract is provided as a part of a prepared formulation (e.g., INFRAGUARD® Powder) which contains Caesalpinia spinosa fruit pod extract/Caesalpinia spinosa fruit extract and Helianthus annuus (sunflower) sprout extract along with other ingredients (such as carriers) like Maltodextrin and water. In some aspects, the INFRAGUARD® powder is incorporated into the composition at a concentration of about 1% which is equivalent to an INFRAGUARD solution at 2%, which has been shown to have beneficial effects.

Helianthus annuus (Sunflower) Sprout Extract

In various aspects, the composition comprises Helianthus annuus (sunflower) sprout extract. In some aspects, the compositions comprise about 0.001% to 5% of Helianthus annuus (sunflower) sprout extract. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% of Helianthus annuus (sunflower) sprout extract. In some aspects, the composition comprises up to 1.0% of Helianthus annuus (sunflower) sprout extract.

In some aspects, the Helianthus annuus (sunflower) sprout extract is provided as a part of a prepared formulation (e.g., INFRAGUARD® Powder) which contains Caesalpinia spinosa fruit pod extract/Caesalpinia spinosa fruit extract and Helianthus annuus (sunflower) sprout extract along with other ingredients (such as carriers) like Maltodextrin and water. In some aspects, the INFRAGUARD® powder is incorporated into the composition at a concentration of about 1% which is equivalent to an INFRAGUARD solution at 2%, which has been shown to have beneficial effects.

### 4. Skin Conditioner

In various aspects, the compositions comprise at least one skin conditioner. In various aspects, the at least one skin conditioner comprises caprylic/capric triglyceride, coconut alkanes, isopropyl isostearate, or any combination thereof.

In various aspects, the at least one skin conditioner at a concentration comprises about 15% to about 35% by weight of the total composition. For instance, in some aspects, the composition comprises at least, at most, exactly, or about 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, or 35% of at least one skin conditioner.

In some aspects, the composition comprises at least two skin conditioners (e.g., selected from caprylic/capric triglyceride, isopropyl isostearate, and coconut alkanes). In some aspects, the composition comprises at least three skin conditioners (e.g., caprylic/capric triglyceride, isopropyl isostearate, and coconut alkanes). In some aspects, the composition comprises a first skin conditioner comprising about 15% to about 25% by weight of the total composition, a second skin conditioner comprising about 1% to 5% by weight of the total composition, and/or a third skin conditioner comprising about 1% to 5% by weight of the total composition. In some aspects, the first skin conditioner comprises caprylic/capric triglyceride. In some aspects, the second skin conditioner comprises isopropyl isostearate. In some aspects, the third skin conditioner comprises coconut alkanes.

### Caprylic/Capric Triglyceride

In some aspects, the composition comprises caprylic/capric triglyceride. Caprylic/capric triglyceride is used as a skin conditioner. In formulations it improves spreadability and smoothness, improving the texture and stability. It is also moisturizing, non-pore clogging and has been shown to improve skin hydration, protect from environmental stressors and reduce fine lines and wrinkles. In some aspects, the composition comprises 10 to 30% by weight of caprylic/capric triglyceride (a skin conditioner). In some aspects, the composition comprises 15 to 25% by weight of caprylic/capric triglyceride. For example, in some aspects the composition comprises at least, at most, exactly, or about 10%, 11%, 12%, 13%, 14%, 15%, 16%, 17%, 18%, 19%, 20%, 20%, 21%, 22%, 23%, 24%, 25%, 26%, 27%, 28%, 29%, or 30% by weight of caprylic/capric triglyceride.

### Isopropyl Isostearate

In some aspects, the composition comprises isopropyl isostearate. Isopropyl isostearate is used as a binder, skin conditioner, and emollient. In some aspects, the composition comprises about 1 to 5% by weight of isopropyl isostearate. For example, in some aspects the composition comprises at least, at most, exactly, or about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of isopropyl isostearate.

### Coconut Alkanes

In some aspects, the composition comprises coconut alkanes. Coconut alkanes is an ingredient derived from the reduction and hydrogenation of fatty acids from coconut oil and is used in skincare products as a skin conditioning agent, solvent and emollient. It enhances the texture and spreadability of formulations and moisturize and condition skin without clogging pores. In some aspects, the composition comprises 1 to 5% by weight of coconut alkanes. In some aspects, the composition comprises 1 to 5% by weight of coconut alkanes. For example, in some aspects the composition comprises at least, at most, exactly, or about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of coconut alkanes.

### 5. Humectant

Humectants are substances that attract and retain moisture from the air or skin. They are used in skincare products to hydrate the skin, hair or nails. In some aspects, the compositions comprise or further comprise one or more humectants. In some aspects, the humectant comprises glycerin. In some aspects, the humectant comprises hyaluronic acid or a derivative thereof (e.g., sodium hyaluronate). In some aspects, the humectant comprises glycerin and/or hyaluronic acid or a derivative thereof (e.g., sodium hyaluronate).

### Glycerin

In some aspects, the composition comprises glycerin. In some aspects, the composition comprises 1 to 5% by weight of glycerin. For example, in some aspects the composition comprises at least, at most, exactly, or about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of glycerin.

### Sodium Hyaluronate

In some aspects, the composition comprises sodium hyaluronate, a derivative of hyaluronic acid used as a humectant and skin conditioner. In some aspects, the composition comprises up to 1% by weight of sodium hyaluronate. In some aspects, the composition comprises at least, at most, exactly, or about 0.001% to 1% of niacinamide. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% sodium hyaluronate.

### 6. Nonaqueous Solvent

In some aspects, the composition comprises or further comprises a nonaqueous solvent. In some aspects, the nonaqueous solvent may be an emollient (e.g., a substance that improves the texture and feel of a cosmetic product on a skin). In some aspects, the nonaqueous solvent comprises C13-C15 alkane, a light emollient that helps provide a pleasant, silky, lightweight texture to the disclosed composition with a non-sticky, non-greasy finish.

Accordingly, in some aspects, the composition comprises C13-15 Alkane. In some aspects, the composition comprises 1 to 5% by weight of C13-15 Alkane. For example, in some aspects the composition comprises at least, at most, exactly, or about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of C13-15 Alkane.

### 7. Surfactant, Emulsifier, Emulsion Stabilizer

In some aspects, the composition comprises or further comprises a surfactant, an emulsifier, an emulsion stabilizer, or any combination thereof. In some instances, the surfactant, emulsifiers and/or emulsion stabilizers comprises polyglyceryl-4 diisostearate/polyhydroxystearate/sebacate, a diester of a mixture of isostearic, polyhydroxystearic and sebacic acids with polyglycerin-4.

Accordingly, in some aspects, the composition comprises polyglyceryl-4 diisostearate/polyhydroxystearate/sebacate. In some aspects, the composition comprises 1 to 5% by weight of polyglyceryl-4 diisostearate/polyhydroxystearate/sebacate. For example, in some aspects the composition comprises at least, at most, exactly, or about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of polyglyceryl-4 diisostearate/polyhydroxystearate/sebacate.

### 8. Additional Ingredients

In some aspects, the composition may comprise one or more additional ingredients that further improve the formulation for topical applications. In some aspects, the one or more additional ingredients comprise another a skin conditioner, another antioxidant, another humectant, another nonaqueous solvent, another emulsifier or emulsion stabilizer, another surfactant, an emollient, a bulking agent, a viscosity controller, an antioxidant, a cleanser, a gelling agent, a film former, a preservative, a buffer, a colorant or any combination thereof. In some aspects, each of these additional ingredients may be present in the composition at concentration of up to 1% by weight. Exemplary additional ingredients that may be used or added to the disclosed compositions (e.g., each at a concentration up to 1%) may include, for instance, sodium chloride, niacinamide, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, citric acid, phytosphingosine, cholesterol, sodium lauroyl lactylate, carbomer, and iron oxides, which are all described herein below.

### Sodium Chloride

In some aspects the composition comprises sodium chloride which is used in skin formulations as a bulking agent and viscosity controller. In some aspects, the composition comprises up to 1% by weight of sodium chloride. In some aspects, the composition comprises at least, at most, exactly, or about 0.001% to 1% of sodium chloride. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% sodium chloride.

### Niacinamide

In some aspects, the composition comprises niacinamide which is used in skin formulations as a smoothing agent. In some aspects, the composition comprises up to 1% by weight of niacinamide. In some aspects, the composition comprises at least, at most, exactly, or about 0.001% to 1% of niacinamide. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% niacinamide.

### Coco-Caprylate/Caprate

In some aspects, the composition comprises coco-caprylate/caprate which is a light emollient ester derived from coconut oil and glycerin. It is used in cosmetics to provide a non-oily, smooth and velvety sensation. In some aspects, the composition comprises up to 1% by weight of coco-caprylate/caprate. In some aspects, the composition comprises at least, at most, exactly, or about 0.001% to 1% of coco-caprylate/caprate. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% coco-caprylate/caprate.

### Polyhydroxystearic Acid

In some aspects, the composition comprises polyhydroxystearic acid, which is a natural, soft, pliable wax derived from castor oil. It is used as a texturing agent, thickener, emollient and film former in cosmetics and as an oil-gelling agent in sunscreen products. In some aspects, the composition comprises up to 1% by weight of polyhydroxystearic acid. In some aspects, the composition comprises at least, at most, exactly, or about 0.001% to 1% of polyhydroxystearic acid. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% polyhydroxystearic acid.

### Polyqlyceryl-3 Polyricinoleate

In some aspects, the composition comprises polyglyceryl-3 polyricinoleate, an emulsifier made from glycerol and fatty acids. It helps improve the skin feel of low viscosity water in oil emulsions. In some aspects, the composition comprises up to 1% by weight of polyglyceryl-3 polyricinoleate. In some aspects, the composition comprises at least, at most, exactly, or about 0.001% to 1% of polyglyceryl-3 polyricinoleate. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% polyglyceryl-3 polyricinoleate.

### Isostearic Acid

In some aspects, the composition comprises isostearic acid. Isostearic acid is fatty acid that acts as a surfactant, cleanser, emollient, stabilizer and conditioner in topical formulations. In some aspects, the composition comprises up to 1% by weight of isostearic acid. In some aspects, the composition comprises at least, at most, exactly, or about 0.001% to 1% of isostearic acid. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% isostearic acid.

### Lecithin

In some aspects, the composition comprises lecithin. Lecithin is a generic term designating a group of yellow-brownish fatty substances occurring in animal and plant tissues. It acts as a surfactant and emulsifier. In some aspects, the composition comprises up to 1% by weight of lecithin. In some aspects, the composition comprises at least, at most, exactly, or about 0.001% to 1% of lecithin. For example, in some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% lecithin. In some aspects, the lecithin is derived from plant sources.

### Ceramides

In some aspects, the composition comprises one or more ceramides. Ceramides are lipid molecules that help maintain integrity and functionality of the skin's natural barrier. They are comprised of a sphingosine and a fatty acid joined by an amide bond. Commercially available ceramides include Ceramide NP (CAS No. 72968-43-5), Ceramide AP (CAS No. 100403-19-8), and Ceramide EOP (CAS No. 627881-96-3). In some aspects, the composition comprises Ceramide NP. In some aspects, the composition comprises Ceramide AP. In some aspects, the composition comprises Ceramide EOP. In some aspects, the composition comprises Ceramide NP and Ceramide EOP. In some aspects, the composition comprises Ceramide NP and Ceramide AP. In some aspects, the composition comprises Ceramide AP and Ceramide EOP. In some aspects, the composition comprises Ceramide NP, Ceramide AP and Ceramide EOP. In various aspects, each ceramide in the composition is present at a concentration of up to 1% by weight of the total composition. For example, the composition comprises up to 1% by weight of Ceramide NP. In some aspects, the composition comprises at least, at most, exactly, or about 0.001% to 1% of Ceramide NP. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% Ceramide NP. In some aspects, the composition comprises up to 1% by weight of Ceramide AP. In some aspects, the composition comprises at least, at most, exactly, or about 0.001% to 1% of Ceramide AP. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% Ceramide AP. In some aspects, the composition comprises up to 1% by weight of Ceramide EOP. In some aspects, the composition comprises at least, at most, exactly, or about 0.001% to 1% of Ceramide EOP. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% Ceramide EOP. In some aspects, the composition comprises up to 1% (e.g., 0.001% to 1%) of Ceramide NP, up to 1% (e.g., 0.001% to 1%) Ceramide AP and up to 1% (e.g., 0.001% to 1%) Ceramide EOP.

### Xanthan Gum

In some aspects, the composition comprises xanthan gum which is an emulsion stabilizer, gel former and viscosity controller. In some aspects, the composition comprises up to 1% by weight of xanthan gum. In some aspects, the composition comprises at least, at most, exactly, or about 0.001% to 1% of xanthan gum. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% xanthan gum.

### Maltodextrin

In some aspects, the composition comprises maltodextrin which acts as an absorbent, binder, emulsion stabilizer, and film former. In some aspects, the composition comprises up to 1% by weight of maltodextrin. In some aspects, the composition comprises at least, at most, exactly, or about 0.001% to 1% of maltodextrin. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% maltodextrin.

### Ethylhexylqlycerin

In some aspects, the composition comprises ethylhexylglycerin, also called octoxyglycerin, which is a skin conditioner and preservative. In some aspects, the composition comprises up to 1% by weight of ethylhexylglycerin. In some aspects, the composition comprises at least, at most, exactly, or about 0.001% to 1% of ethylhexylglycerin. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% ethylhexylglycerin.

### Caprylyl Glycol

In some aspects, the composition comprises caprylyl glycol, also referred to as 1,2-octanediol. Capryl glycol is used as a skin conditioning agent and also has antimicrobial (preservative) properties. In some aspects, the composition comprises up to 1% by weight of capryl glycol. In some aspects, the composition comprises at least, at most, exactly, or about 0.001% to 1% of capryl glycol. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% capryl glycol.

### Hexylene Glycol

In some aspects, the composition comprises hexylene glycol, also known as 2-methyl-2,4-pentanediol, which acts as a surfactant, emulsifier, and viscosity reducing agent. In some aspects, the composition comprises up to 1% by weight of hexylene glycol. In some aspects, the composition comprises at least, at most, exactly, or about 0.001% to 1% of hexylene glycol. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% hexylene glycol.

### Phenoxyethanol

In some aspects, the composition comprises phenoxyethanol, a preservative. In some aspects, the composition comprises up to 1% by weight of phenoxyethanol. In some aspects, the composition comprises at least, at most, exactly, or about 0.001% to 1% of phenoxyethanol. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% phenoxyethanol.

### Sodium Benzoate

In some aspects, the composition comprises sodium benzoate, a preservative. In some aspects, the composition comprises up to 1% by weight of sodium benzoate. In some aspects, the composition comprises at least, at most, exactly, or about 0.001% to 1% of sodium benzoate. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% sodium benzoate.

### Potassium Sorbate

In some aspects, the composition comprises potassium sorbate, a preservative. In some aspects, the composition comprises up to 1% by weight of potassium sorbate. In some aspects, the composition comprises at least, at most, exactly, or about 0.001% to 1% of potassium sorbate. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% potassium sorbate.

### Citric Acid

In some aspects, the composition comprises citric acid, a buffer that also has exfoliating and antioxidant properties. In some aspects, the composition comprises up to 1% by weight of citric acid. In some aspects, the composition comprises at least, at most, exactly, or about 0.001% to 1% of citric acid. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% citric acid.

### Phytosphingosine

In some aspects, the composition comprises phytosphingosine. Phytosphingosine is used as a skin conditioner for its ability to help skin barrier function. In some aspects, the composition comprises up to 1% by weight of phytosphingosine. In some aspects, the composition comprises at least, at most, exactly, or about 0.001% to 1% of phytosphingosine. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% phytosphingosine.

### Cholesterol

In some aspects, the composition comprises cholesterol. Cholesterol serves as a skin conditioning agent and emulsifier as it supports the skin's natural barrier function and improves moisture content of topical formulations. In some aspects, the composition comprises up to 1% by weight of cholesterol. In some aspects, the composition comprises at least, at most, exactly, or about 0.001% to 1% of cholesterol. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% cholesterol.

### Sodium Lauroyl Lactylate

In some aspects, the composition comprises sodium lauroyl lactylate which is a natural surfactant and emulsifier. It is a sodium salt of lauric acid and lactic acid and is derived from coconut oil and milk. In some aspects, the composition comprises up to 1% by weight of sodium lauroyl lactylate. In some aspects, the composition comprises at least, at most, exactly, or about 0.001% to 1% of sodium lauroyl lactylate. In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% sodium lauroyl lactylate.

### Carbomer

In some aspects, the composition comprises carbomer (polyacrylic acid, CAS No. 600-07-7) which is a viscosity controller. In some aspects, the composition comprises up to 1% by weight of carbomer (polyacrylic acid). In some aspects, the composition comprises at least, at most, exactly, or about 0.001% to 1% of carbomer (polyacrylic acid). In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% carbomer (polyacrylic acid).

### Iron Oxides

In some aspects, the composition comprises one or more iron oxides which are used as colorants. Suitable iron oxides that may be incorporated into the composition herein include CI 77492 (CAS No. 51274-00-1), CI 77491 (CAS No. 1309-37-1) and CI 77499 (CAS No. 1317-61-9). In some aspects, the composition comprises up to 1% by weight of iron oxide (e.g., CI 77492, 77491, and/or CI 77499). In some aspects, the composition comprises at least, at most, exactly, or about 0.001% to 1% of iron oxide (e.g., CI 77492, 77491, and/or CI 77499). In some aspects, the composition comprises at least, at most, exactly, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0% of iron oxide (e.g., CI 77492, 77491, and/or CI 77499).

### B. Other Ingredients

Other ingredients are contemplated herein. In addition to the ingredients listed above, the compositions disclosed herein may comprise agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the composition of the present disclosure. In some aspects, additives may be biocompatible, without being harsh, abrasive, and/or allergenic.

In certain aspects, compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any suitable organic or inorganic acid may be used. In some aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some aspects, compositions disclosed herein may comprise about 5% to about 99%, about 10%, about 95%, or about 15% to about 90% total amount of one or more acidifying agents by total weight of the composition.

In certain aspects, compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any suitable organic or inorganic base can be used. In some aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some aspects, compositions disclosed herein may comprise about 5% to about 99%, about 10%, about 95%, or about 15% to about 90% total amount of one or more alkalizing agents by total weight of the composition.

In certain aspects, compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition. In some aspects, compositions disclosed herein may comprise about 5% to about 99%, about 10%, about 95%, or about 15% to about 90% total amount of one or more antioxidants by total weight of the composition.

In certain aspects, compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any suitable organic or inorganic buffer can be used. In some aspects, compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, compositions disclosed herein may comprise about 5% to about 99%, about 10%, about 95%, or about 15% to about 90% total amount of one or more buffering agents by total weight of the composition. In some aspects, the amount of one or more buffering agents may depend on the desired pH level of a composition. In some aspects, compositions disclosed herein may have a pH of about 6 to about 9. In some aspects, compositions disclosed herein may have a pH greater than about 8, greater than about 7.5, greater than about 7, greater than about 6.5, or greater than about 6.

In certain aspects, compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin® (methylparaben), Nipasol™ (propylparaben), isopropyl alcohol, and any combination thereof. In some aspects, any suitable preservative can be used. In some aspects, compositions disclosed herein may comprise at least 5%, at least 10%, at least 20%, at least 25%, at least 30%, at least 35%, at least 40%, at least 45%, or at least 50% total amount of one or more preservatives by total weight of the composition. In some aspects, compositions disclosed herein may comprise about 5% to about 99%, about 10%, about 95%, or about 15% to about 90% total amount of one or more preservatives by total weight of the composition.

In certain aspects, compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, compositions disclosed herein may comprise at least 5%, at least 10%, at least 20%, at least 25%, at least 30%, at least 35%, at least 40%, at least 45%, or at least 50% total amount of one or more surface-acting reagents or detergents by total weight of the composition.

In some aspects, compositions disclosed herein may comprise about 5% to about 99%, about 10%, about 95%, or about 15% to about 90% total amount of one or more surface-acting reagents or detergents by total weight of the composition.

In certain aspects, compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, compositions disclosed herein may comprise at least 5%, at least 10%, at least 20%, at least 25%, at least 30%, at least 35%, at least 40%, at least 45%, or at least 50% total amount of one or more stabilizers by total weight of the composition. In some aspects, compositions disclosed herein may comprise about 5% to about 99%, about 10%, about 95%, or about 15% to about 90% total amount of one or more stabilizers by total weight of the composition.

In some aspects, compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, compositions disclosed herein may comprise at least 5%, at least 10%, at least 20%, at least 25%, at least 30%, at least 35%, at least 40%, at least 45%, or at least 50% total amount of one or more tonicity modifiers by total weight of the composition. In some aspects, compositions disclosed herein may comprise about 5% to about 99%, about 10%, about 95%, or about 15% to about 90% total amount of one or more tonicity modifiers by total weight of the composition.

In certain aspects, compositions described herein may be an aqueous suspension comprising one or more polymers as suspending agents. In some aspects, polymers that may comprise compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, compositions disclosed herein may comprise about 5% to about 99%, about 10%, about 95%, or about 15% to about 90% total amount of polymers as suspending agent(s) by total weight of the composition.

In certain aspects, compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on a skin surface. In some aspects, compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, hypermellose, plullulan, pectin Ghatti gum, guar gum, hectorite, lactose, sucrose, maltodextrin, mannitol, sorbitol, honey, maize starch, wheat starch, rice starch, potato starch, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In some aspects, one or more of the ingredients disclosed herein may be from an animal source, a vegetarian source, or a vegan source. In some aspects, the disclosed compositions comprise or consists essentially of ingredients from vegan and/or vegetarian friendly sources. In some aspects, the one or more of the ingredients disclosed herein are derived from non-GMO sources. In some aspects, all the ingredients disclosed herein are derived from non-GMO sources. In some aspects the compositions disclosed herein do not comprise any ingredients that contain wheat, gluten, soy, dairy products and/or nuts. In some aspects, the compositions disclosed herein do not comprise artificial colors and/or preservatives.

It is contemplated that the compositions of the present disclosure can include any amount of the ingredients discussed in this specification. The compositions can also include any number of combinations of additional ingredients described throughout this specification. For example, the compositions can also be mixed with other active agents that do not impair the desired action, or with materials that supplement the desired action. The concentrations of any ingredient within the compositions can vary. In non-limiting aspects, for example, the compositions can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in each composition.

### C. Topical Formulations

The compositions of the present disclosure can be formulated in various forms suitable for topical delivery. Non-limiting examples include semi-solid compositions, lotions, liquid suspensions, emulsions, creams, gels, ointments, pastes, aerosol sprays, aerosol foams, non-aerosol sprays, non-aerosol foams, films and sheets. Semi-solid compositions include ointments, pastes and creams. The compositions can be impregnated in gauzes, bandages, or other skin dressing materials. In some embodiments, the compositions are semi-solid compositions. In some embodiments, the compositions are ointments. In other embodiments, the compositions are gels. In still other embodiments, the compositions are liquid suspensions. In some embodiments, the compositions are topical serums.

Compositions suitable for use in context of the present disclosure may include compositions wherein the active ingredients can be contained in an amount effective to achieve the intended purpose. In some aspects, a therapeutically effective amount means an amount of active ingredients effective to prevent, slow, alleviate or ameliorate symptoms of sun-induced skin damage, skin aging, or skin sensitivity.

Methods for preparing topical formulations are well understood in the art. In some aspects, the compositions of the present disclosure may be manufactured by processes well known in the art, e.g., by means of conventional mixing, dissolving, granulating, dragee-making, levigating, emulsifying, encapsulating, entrapping or lyophilizing processes. In certain aspects, compositions for use in accordance with the present disclosure may be formulated in conventional manner using one or more physiologically acceptable carriers comprising ingredients, described above, which facilitate processing of the active ingredients into preparations.

The compositions of the present disclosure can be packaged in any package configuration suitable for topical products. Non-limiting examples include bottles, bottles with pumps, tubes (aluminum, plastic, or laminated), jars, non-aerosol pump sprayers, aerosol containers, pouches, and packets. The package can be configured for single-dose or multiple-dose administration. The enclosed compositions can be provided in kits, for example, including component parts that can be assembled for use. A kit may include a plurality of containers, each container holding one or more unit dose of the one or more active agents. The containers are preferably adapted for the desired mode of administration, including but not limited to serums, ointments, creams, and/or lotions for topical administration.

### D. Exemplary Compositions

As noted, in various aspects, the compositions herein may comprise zinc oxide, an antioxidant (e.g., ascorbic acid or a derivative thereof such as sodium ascorbyl phosphate (SAP)), and at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract). Accordingly, the compositions may comprise zinc oxide, sodium ascorbyl phosphate (SAP), and at least one plant extract selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract. In some aspects, the composition may comprise 16.1% zinc oxide, up to 1% of sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract.

In various aspects, the composition may comprise or further comprise a skin conditioner. Accordingly, the composition may comprise zinc oxide, an antioxidant (e.g., ascorbic acid or a derivative thereof such as sodium ascorbyl phosphate (SAP)), at least one skin conditioner (e.g., caprylic/capric triglyceride, coconut alkanes, isopropyl isostearate, or any combination thereof), and at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract). In some aspects, the composition may comprise one or more skin conditioners (e.g., caprylic/capric triglyceride, coconut alkanes, and/or isopropyl isostearate) which together comprise 15 to 35% of the total composition. Accordingly, in some aspects, the composition may comprise 16.1% zinc oxide, 15 to 25% of caprylic/capric triglyceride, 1 to 5% isopropyl isostearate, 1 to 5% coconut alkanes, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract.

In various aspects, the composition may comprise or further comprise a humectant. In some aspects, the humectant is at a concentration of about 1 to 5% by weight of the total composition. Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and at least one humectant (e.g., glycerin). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% glycerin.

In various aspects, the composition may comprise or further comprise at least one skin conditioner and at least one humectant. Accordingly, the composition may comprise zinc oxide, an antioxidant (e.g., ascorbic acid or a derivative thereof such as sodium ascorbyl phosphate (SAP)), at least one skin conditioner (e.g., caprylic/capric triglyceride, coconut alkanes, isopropyl isostearate, or any combination thereof), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and at least one humectant (e.g., glycerin). In some aspects, therefore the composition may comprise 16.1% zinc oxide, 15 to 25% of caprylic/capric triglyceride, 1 to 5% isopropyl isostearate, 1 to 5% coconut alkanes, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% glycerin.

In various aspects, the composition may comprise or further comprise a nonaqueous solvent (e.g., C13-C15 alkane). In some aspects, the nonaqueous solvent is at a concentration of about 1 to 5% by weight of the total composition. Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and at least one nonaqueous solvent (e.g., C13-C15 alkane). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% C13-C15 alkane.

In various aspects, the composition may comprise or further comprise at least one skin conditioner (e.g., caprylic/capric triglyceride, coconut alkanes, isopropyl isostearate, or any combination thereof) and a nonaqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one skin conditioner (e.g., caprylic/capric triglyceride, coconut alkanes, isopropyl isostearate, or any combination thereof), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and at least one nonaqueous solvent (e.g., C13-C15 alkane). In some aspects, therefore the composition may comprise 16.1% zinc oxide, 15 to 25% of caprylic/capric triglyceride, 1 to 5% isopropyl isostearate, 1 to 5% coconut alkanes, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% C13-C15 alkane.

In various aspects, the composition may comprise or further comprise at least one skin conditioner (e.g., caprylic/capric triglyceride, coconut alkanes, isopropyl isostearate, or any combination thereof), a humectant (e.g., glycerin) and a nonaqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one skin conditioner (e.g., caprylic/capric triglyceride, coconut alkanes, isopropyl isostearate, or any combination thereof), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), at least one humectant (e.g., glycerin) and at least one nonaqueous solvent (e.g., C13-C15 alkane). In some aspects, therefore the composition may comprise 16.1% zinc oxide, 15 to 25% of caprylic/capric triglyceride, 1 to 5% isopropyl isostearate, 1 to 5% coconut alkanes, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% glycerin, and 1 to 5% C13-C15 alkane.

In various aspects, the composition may comprise or further comprise surfactant, emulsifier, emulsion stabilizer, or any combination thereof (e.g., polyglyceryl-4 diisostearate/polyhydroxystearate/sebacate). In some aspects, the surfactant, emulsifier, emulsion stabilizer, or any combination thereof is at a concentration of about 1 to 5% by weight of the total composition. Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and at least one surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% polyglyceryl-4 diisostearate/polyhydroxystearate/sebacate.

In various aspects, the composition may comprise or further comprise at least one skin conditioner (e.g., caprylic/capric triglyceride, coconut alkanes, isopropyl isostearate, or any combination thereof) and a surfactant, emulsifier, emulsion stabilizer, or any combination thereof (e.g., polyglyceryl-4 diisostearate/polyhydroxystearate/sebacate). Accordingly, the composition may comprise zinc oxide, an antioxidant (e.g., ascorbic acid or a derivative thereof such as sodium ascorbyl phosphate (SAP)), at least one skin conditioner (e.g., caprylic/capric triglyceride, coconut alkanes, isopropyl isostearate, or any combination thereof), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, 15 to 25% of caprylic/capric triglyceride, 1 to 5% isopropyl isostearate, 1 to 5% coconut alkanes, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate) and a humectant (e.g., glycerin). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), a humectant (e.g., glycerin) and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% glycerin, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate) and a nonaqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), a nonaqueous solvent (e.g., C13-C15 alkane), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% C13-C15 alkane, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate), at least one skin conditioner (e.g., caprylic/capric triglyceride, coconut alkanes, isopropyl isostearate, or any combination thereof), and a humectant (e.g., glycerin). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), at least one skin conditioner (e.g., caprylic/capric triglyceride, coconut alkanes, isopropyl isostearate, or any combination thereof), a humectant (e.g., glycerin), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), 15 to 25% of caprylic/capric triglyceride, 1 to 5% isopropyl isostearate, 1 to 5% coconut alkanes, up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% glycerin, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate), at least one skin conditioner (e.g., caprylic/capric triglyceride, coconut alkanes, isopropyl isostearate, or any combination thereof), and a non-aqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), at least one skin conditioner (e.g., caprylic/capric triglyceride, coconut alkanes, isopropyl isostearate, or any combination thereof), a nonaqueous solvent (e.g., C13-C15 alkane), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), 15 to 25% of caprylic/capric triglyceride, 1 to 5% isopropyl isostearate, 1 to 5% coconut alkanes, up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% C13-C15 alkane, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate), a humectant (e.g., glycerin), and a nonaqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), a humectant (e.g., glycerin), a nonaqueous solvent (e.g., C13-C15 alkane), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% glycerin, 1 to 5% C13-C15 alkane, and 1 to 5% polyglyceryl-4 diisostearate/polyhydroxystearate/sebacate.

In various aspects, the composition may comprise or further comprise a surfactant, emulsifier, emulsion stabilizer, or any combination thereof (e.g., polyglyceryl-4 diisostearate/polyhydroxystearate/sebacate), at least one skin conditioner (e.g., caprylic/capric triglyceride, coconut alkanes, isopropyl isostearate, or any combination thereof), a humectant (e.g., glycerin), and a non-aqueous solvent (e.g., C13-C15 alkane). Accordingly, the composition may comprise zinc oxide, an antioxidant (e.g., ascorbic acid or a derivative thereof such as sodium ascorbyl phosphate (SAP)), at least one plant extract (e.g., selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract), at least one skin conditioner (e.g., caprylic/capric triglyceride, coconut alkanes, isopropyl isostearate, or any combination thereof), a humectant (e.g., glycerin), a nonaqueous solvent (e.g., C13-C15 alkane), and a surfactant, emulsifier, emulsion stabilizer, or any combination thereof (e.g., polyglyceryl-4 diisostearate/polyhydroxystearate/sebacate). In some aspects, therefore the composition may comprise 16.1% zinc oxide, up to 1% sodium ascorbyl phosphate (SAP), 15 to 25% of caprylic/capric triglyceride, 1 to 5% isopropyl isostearate, 1 to 5% coconut alkanes, up to 1% of Camellia sinensis (green tea) leaf extract, up to 1% of Opuntia ficus-indica stem (prickly pear) extract, up to 1% of Caesalpinia spinosa (spiny holdback) fruit pod extract, up to 1% of Helianthus annuus (sunflower) sprout extract, 1 to 5% glycerin, 1 to 5% C13-C15 alkane, and 1 to 5% polyglyceryl-4 diisostearate/polyhydroxystearate/sebacate.

In various aspects, the compositions herein may comprise one or more additional ingredients selected from sodium chloride, niacinamide, sodium hyaluronate, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, citric acid, phytosphingosine, cholesterol, sodium lauroyl lactylate, carbomer, wherein each of the additional ingredients are present at a concentration of up to 1% by weight of the total composition.

In various aspects, the compositions herein further comprise one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more of sodium chloride, niacinamide, sodium hyaluronate, coco-caprylate/caprate, polyhydroxystearic acid, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, and citric acid, each at a concentration up to 1% by weight of the total composition.

In some aspects, the compositions comprise sodium chloride, niacinamide, sodium hyaluronate, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, citric acid, phytosphingosine, cholesterol, sodium lauroyl lactylate, and carbomer, each at a concentration up to 1% by weight of the total composition.

In various aspects, the compositions may further comprise one or more colorants (e.g., iron oxides) at a concentration up to 1% by weight of the total composition.

As an illustrative, non-limiting example, Table 1 is provided herein with an exemplary composition according to aspects of the present disclosure.

TABLE 1

| Active Ingredient | CAS Number | Percentage | Origin | Function |
|---|---|---|---|---|
| Zinc Oxide | 1314-13-2 | 16.1 | Mineral | UV Filter |

| | | Percentage | | |
|---|---|---|---|---|
| Inactive Ingredients | CAS Number | Range | Origin | Function |
| Water (Aqua) | 7732-18-5 | QS to 100 | Mineral | Solvent |
| Caprylic/Capric Triglyceride | 73398-61-5 | 15-25 | Plant | Skin Conditioner |
| Isopropyl Isostearate | 68171-33-5 | 1-5 | Synthetic, | Binder, Skin |
| | | | Plant | Conditioner, Emollient |
| Polyglyceryl-4 | N/A | 1-5 | Vegetable | Surfactant, Emulsifier, |
| Diisostearate/ | | | | Emulsion Stabilizer |
| Polyhydroxystearate/Sebacate | | | | |
| Coconut Alkanes | 112-40-3 | 1-5 | Vegetable | Skin Conditioner, |
| | | | | Emollient |
| C13-15 Alkane | 3891-98-3 | 1-5 | Plant | Solvent |
| | | | Sugars | |
| Glycerin | 56-81-5 | 1-5 | Plant | Humectant |
| Sodium Chloride | 7647-14-5 | ≤1 | Mineral | Bulking, Viscosity |
| | | | | Controller |
| Niacinamide | 98-92-0 | ≤1 | Synthetic | Smoothing |
| Sodium Hyaluronate | 9067-32-7 | ≤1 | Fermentation | Humectant, Skin |
| | | | | Conditioner |
| Camellia sinensis (Green Tea) | 84650-60-2 | ≤1 | Plant | Antioxidant, Skin |
| Leaf Extract | | | | Conditioner |
| Opuntia ficus-indica Stem | 90082-21-6 | ≤1 | Vegetable | Skin Conditioner |
| Extract | | | | |
| Caesalpinia spinosa Fruit Pod | 39300-88-4 | ≤1 | Vegetable | Skin Conditioner |
| Extract | | | | |
| Helianthus annuus (Sunflower) | 8001-21-6 | ≤1 | Vegetable | Skin Conditioner |
| Sprout Extract | | | | |
| Coco-Caprylate/Caprate | 95912-86-0 | ≤1 | Vegetable | Skin Conditioner, |
| | | | | Emollient |
| Polyhydroxystearic acid | 27924-99-8 | ≤1 | Plant | Surfactant, Emulsifier |
| Polyglyceryl-3 Polyricinoleate | 29894-35-7; | ≤1 | Plant | Surfactant, Emulsifier |
| | 235783-76-3 | | | |
| Isostearic acid | 30399-84-9 | ≤1 | Plant | Surfactant, Cleanser, |
| | | | | Emulsifier |
| Lecithin | 8002-43-5 | ≤1 | Plant | Skin Conditioner, |
| | | | | Emollient, Surfactant, |
| | | | | Emulsifier |
| Ceramide NP | 72968-43-5 | ≤1 | Vegetable | Skin Conditioner |
| Ceramide AP | 100403-19-8 | ≤1 | Vegetable | Skin Conditioner |
| Ceramide EOP | 627881-96-3 | ≤1 | Vegetable | Skin Conditioner |
| Sodium Ascorbyl Phosphate | 66170-10-3 | ≤1 | Synthetic | Antioxidant |
| Xanthan Gum | 11138-66-2 | ≤1 | Biofermentation | Emulsion Stabilizer, Gel |
| | | | | former, Viscosity |
| | | | | Controller |
| Maltodextrin | 9050-36-6 | ≤1 | Vegetable | Absorbent, Binder, |
| | | | | Emulsion Stabilizer, Film |
| | | | | Former |
| Ethylhexylglycerin | 70445-33-9 | ≤1 | Synthetic | Skin Conditioner |
| Caprylyl Glycol | 1117-86-8 | ≤1 | Synthetic | Skin Conditioner, |
| | | | | Emollient |
| Hexylene Glycol | 107-41-5 | ≤1 | Synthetic | Skin Conditioner, |
| | | | | Surfactant, Emulsifier |
| Phenoxyethanol | 122-99-6 | ≤1 | Synthetic | Preservative |
| Sodium Benzoate | 532-32-1 | ≤1 | Synthetic | Preservative |
| Potassium Sorbate | 24634-61-5 | ≤1 | Synthetic | Preservative |
| Citric Acid | 77-92-9/ | ≤1 | Synthetic | Buffer |
| | 5949-29-1 | | | |
| Phytosphingosine | 13552-11-9 | ≤1 | Vegetable | Skin Conditioner |
| Cholesterol | 80356-14-5 | ≤1 | Semi- | Skin Conditioner, |
| | | | Synthetic | Emollient |
| Sodium lauroyl lactylate | 13557-75-0 | ≤1 | Natural | Surfactant, Emulsifier |
| Carbomer | 600-07-7 | ≤1 | Synthetic | Emulsion Stabilizer, Gel |
| | | | | former, Viscosity |
| | | | | Controller |

May Contain+/−

| | | | | |
|---|---|---|---|---|
| Iron oxides (CI 77492) | 51274-00-1 | ≤1 | Mineral, | Colorant |
| | | | Synthetic | |
| Iron oxides (CI 77491) | 1309-37-1 | ≤1 | Mineral, | Colorant |
| | | | Synthetic | |
| Iron oxides (CI 77499) | 1317-61-9 | ≤1 | Mineral, | Colorant |
| | | | Synthetic | |

### III. Method of Use

### A. Sun Protection

In some aspects, the current disclosure encompasses a method of protecting a skin surface from sun exposure in a subject in need thereof, the method comprising topically applying a composition disclosed herein to the skin surface of the subject.

In various aspects, for use in methods of protecting a skin surface from sun exposure, the compositions disclosed herein may be formulated to have a sun protectant factor (SPF) of at least 25, at least 30, at least 35, at least 40, at least 45, at least 50, at least 55, at least 60, at least 65, at least 70, at least 75, at least 80, at least 85, at least 90, at least 95, or at least 100. In some aspects, the compositions herein are formulated to have a sun protectant factor (SPF) of about 30 to 40. In some aspects, the compositions herein are formulated to have a static sun protectant factor (SPF) of about 30 to 40. In some aspects, the compositions herein are formulated to have a sun protectant factor (SPF) of at least 30. In some aspects, the compositions herein are formulated to have a static sun protectant factor (SPF) of at least 30.

SPF is determined using standard methods such as, for example FSTI (Florida Suncare Testing Inc) SOP #2021-01, Static SPF testing and SOP #2021-02 Water Resistant SPF testing, which are described herein. In various aspects, SPF is calculated by determining a subject's “minimal erythemal dose” (MED) in the presence or absence of the therapeutic composition, as described herein. The minimal erythema dose (MED) is the smallest UV dose that produces perceptible redness of the skin (erythema) with clearly defined borders at 16 to 24 hours after UV exposure. The MED for unprotected skin (MEDu) is determined on a test site that does not have sunscreen applied. The MED for protected skin (MEDp) is determined on a test site that has sunscreen applied. SPF is therefore determined as the relationship between the minimal erythemal dose in the presence of the therapeutic composition (eg., MEDp) and the minimal erythemal dose in the absence of the therapeutic composition (e.g., MEDu) according to the following equation:

$$\frac{M E {D}_{p}}{M E {D}_{u}} = S P F \qquad ( Equation 1 )$$

In some aspects, the MED is determined by administering a series of 5 UV radiation doses, expressed as Joules/square meter, progressively increasing in increments of either 15%, 20%, or 25% based on a product's target SPF as specified in OTC Monograph MO20(e)UV exposure. For example, if the SPF of a product is expected to be <8, the 5 UV radiation doses may be: 0.64×, 0.80×, 1.00×, 1.25× or 1.56×; if the SPF is expected to be between 8 and 15, the 5 UV radiation doses may be 0.69×, 0.83×, 1.00×, 1.20× and 1.44×; if the SPF is expected to be >15, the 5 UV doses may be 0.76×, 0.87×, 1.00×, 1.15× and 1.32×. Subjects can be exposed to the 5 UV radiation doses applied to skin contacted with or not contacted with the compositions herein and then evaluated 16 to 24 hours after completion of the UV doses for evaluation of the MED for all sites.

Further details for determining MED and the corresponding SPF of the disclosed compositions are provided in Over-the-Counter Monograph M020: Sunscreen Drug Products for Over-the-Counter Human Use as published by the FDA on Sep. 24, 2021, which is incorporated herein by reference in its entirety.

In various aspects, methods of protecting from sun exposure may comprise ameliorating and/or preventing one or more symptoms associated with sun or UV exposure including but not limited to sunburn, sun rash, itching, pain, blistering, allodynia (hypersensitivity), skin redness (erythema), or any other form of discomfort.

In various aspects, the disclosed compositions do not cause additional skin irritation after repeated use (e.g., as evidenced by a Repeated Insult Patch Test). In various aspects, the disclosed compositions may help firm the look of skin, hydrate and/or shield skin from visible effects of blue light. In various aspects, the disclosed compositions may provide natural, physical protection against UVA/UVB rays in a reef-friendly form, especially compared to compositions that do not comprise zinc oxide. In various aspects, the disclosed compositions may be gentle on skin and be effective longer, especially compared to other compositions that do not comprise sodium ascorbyl phosphate (SAP). In various aspects, the disclosed compositions hydrate and firm skin and help shield skin against visible effects of blue light, especially compared to other compositions that do not comprise botanical extracts (e.g., Camellia sinensis (green tea) leaf extract, Opuntia ficus-indica stem (prickly pear) extract, Caesalpinia spinosa (spiny holdback) fruit pod extract, and Helianthus annuus (sunflower) sprout extract).

### B. Administration

In some aspects, the disclosed composition may be administered in various dosages and may be suitably administered in a topical formulation. In various aspects, the disclosed composition may be administered to a face of a subject (e.g., as a facial topical serum or facial serum). Moreover, the composition may exhibit an effective amount in an administration range of approximately 1 to 5 ounces (oz). The composition may be administered at various doses and in various ways, for example, once a day or several times by dividing daily doses, depending on a subject's body weight, age, sex, health condition, a diet, administration time, an administration method, and the severity of a disease or level of protection from sun desired. In some aspects, administration of a disclosed composition as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, months, 6 months, 7 months, 8 months, 9 months, 10 months, 11 months, 12 months, or more. In some aspects, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. For example, the period of administration of the disclosed composition followed by a period where administration is stopped is repeated for a determined or undetermined period of time. In some aspects, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some aspects, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, months, 6 months, 7 months, 8 months, 9 months, 10 months, 11 months, 12 months, or more.

In some aspects, the composition is for topical administration to a skin surface at least, at most, or about 5 min, 10 min, 20 min, 30 min, 40 min, 50 min, 60 min, 70 min, 80 min, 90 min, 100 min, 110 min, 120 min, 130 min, 140 min, 150 min, 160 min, 170 min, 180 min, 190 min, 200 min, 210 min, 220 min, 230 min, 240 min, 260 min, 270 min, 280 min, 290 min, or 300 min prior to an exposure of the skin surface to a UV radiation source or other skin irritant. In some aspects, the composition is for administration at least, at most, or about 1 hour, up to 2 hours, up to 3 hours, up to 4 hours, or up to 5 hours prior to an exposure of the skin surface to a UV radiation source (e.g., sun). In some aspects, the composition is for administration at least 15 minutes before sun exposure. In various aspects, the method further comprises reapplying the composition (e.g., after exposure to a UV radiation source, after exposure to water, or after a period of time). In some aspects, the composition is formulated for reapplication 5 min, 10 min, 20 min, 30 min, 40 min, 50 min, 60 min, 70 min, 80 min, 90 min, 100 min, 110 min, 120 min, 130 min, 140 min, 150 min, 160 min, 170 min, 180 min, 190 min, 200 min, 210 min, 220 min, 230 min, 240 min, 260 min, 270 min, 280 min, 290 min, or 300 min after a first application and/or after exposure to a UV source and/or after exposure to water. In some aspects, the composition is formulated for reapplication 2 hours after a first application and/or after exposure to a UV source and/or after exposure to water. In some aspects, the composition may be reapplied to a skin surface once, twice, three, or four times an hour. In some aspects, the composition may be reapplied to a skin surface once, twice, three, or four times a day. In some aspects, the composition may be reapplied to a skin surface once, twice, three, or four times a week. In some aspects, the composition may be reapplied to a skin surface once, twice, three, or four a month. In some aspects, the composition may be reapplied to a skin surface once, twice, three, or four times a year. In various aspects, the skin surface may be on or near the face of a subject (e.g., on or near the cheeks, forehead, nose, chin, neck, ears, and the like).

In some aspects, the subject is from 2 years of age to 12 years of age, or 12 years of age through 21 years of age, 21 years of age to 31 years of age, 31 years of age to 41 years of age, 41 years of age to 51 years of age, 51 years of age to 61 years of age, 61 years of age to 71 years of age, 71 years of age to 81 years of age, 81 years of age to 91 years of age, 91 years of age to 101 years of age or more. In some aspects, the subject is an adult subject. In some aspects, the subject is a non-human animal. In some aspects, the subject is a human.

As with any sunscreen product, the effectiveness of the disclosed compositions depends on various factors, including the subject's age, application regimen, genetics, pre-existing skin sensitivities and conditions, lifestyle choices, and other pre-existing health conditions. Given these individual variations, the use cases presented herein should be considered as general guidance rather than definitive outcomes, as responses may differ from person to person.

Having described several embodiments, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

It is to be understood that the specific order or hierarchy of steps in the methods depicted throughout this disclosure are instances of example approaches and can be rearranged while remaining within the disclosed subject matter. For instance, any of the operations discussed throughout this disclosure may be omitted, repeated, performed in parallel, performed in a different order, and/or combined with any other of the operations discussed throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of specific implementations. Functionality may be separated or combined differently in various implementations of the disclosure or described with different terminology. These and other variations, modifications, additions, and improvements may fall within the scope of the disclosure as defined in the claims that follow.

### EXAMPLES

### Example 1: Preparation of Sun Protectant Composition

A sun protectant composition comprising the components listed in Table 2, below, was prepared.

TABLE 2

| Active Ingredient | CAS Number | Percentage | Origin | Function |
|---|---|---|---|---|
| Zinc Oxide | 1314-13-2 | 16.1 | Mineral | UV Filter |

| | | Percentage | | |
|---|---|---|---|---|
| Inactive Ingredients | CAS Number | Range | Origin | Function |
| Water (Aqua) | 7732-18-5 | QS to 100 | Mineral | Solvent |
| Caprylic/Capric Triglyceride | 73398-61-5 | 15-25 | Plant | Skin Conditioner |
| Isopropyl Isostearate | 68171-33-5 | 1-5 | Synthetic, | Binder, Skin |
| | | | Plant | Conditioner, Emollient |
| Polyglyceryl-4 | N/A | 1-5 | Vegetable | Surfactant, Emulsifier, |
| Diisostearate/ | | | | Emulsion Stabilizer |
| Polyhydroxystearate/Sebacate | | | | |
| Coconut Alkanes | 112-40-3 | 1-5 | Vegetable | Skin Conditioner, |
| | | | | Emollient |
| C13-15 Alkane | 3891-98-3 | 1-5 | Plant | Solvent |
| | | | Sugars | |
| Glycerin | 56-81-5 | 1-5 | Plant | Humectant |
| Sodium Chloride | 7647-14-5 | ≤1 | Mineral | Bulking, Viscosity |
| | | | | Controller |
| Niacinamide | 98-92-0 | ≤1 | Synthetic | Smoothing |
| Sodium Hyaluronate | 9067-32-7 | ≤1 | Fermentation | Humectant, Skin |
| | | | | Conditioner |
| Camellia sinensis (Green Tea) | 84650-60-2 | ≤1 | Plant | Antioxidant, Skin |
| Leaf Extract | | | | Conditioner |
| Opuntia ficus-indica Stem | 90082-21-6 | ≤1 | Vegetable | Skin Conditioner |
| Extract | | | | |
| Caesalpinia spinosa Fruit Pod | 39300-88-4 | ≤1 | Vegetable | Skin Conditioner |
| Extract | | | | |
| Helianthus annuus (Sunflower) | 8001-21-6 | ≤1 | Vegetable | Skin Conditioner |
| Sprout Extract | | | | |
| Coco-Caprylate/Caprate | 95912-86-0 | ≤1 | Vegetable | Skin Conditioner, |
| | | | | Emollient |
| Polyhydroxystearic acid | 27924-99-8 | ≤1 | Plant | Surfactant, Emulsifier |
| Polyglyceryl-3 Polyricinoleate | 29894-35-7; | ≤1 | Plant | Surfactant, Emulsifier |
| | 235783-76-3 | | | |
| Isostearic acid | 30399-84-9 | ≤1 | Plant | Surfactant, Cleanser, |
| | | | | Emulsifier |
| Lecithin | 8002-43-5 | ≤1 | Plant | Skin Conditioner, |
| | | | | Emollient, Surfactant, |
| | | | | Emulsifier |
| Ceramide NP | 72968-43-5 | ≤1 | Vegetable | Skin Conditioner |
| Ceramide AP | 100403-19-8 | ≤1 | Vegetable | Skin Conditioner |
| Ceramide EOP | 627881-96-3 | ≤1 | Vegetable | Skin Conditioner |
| Sodium Ascorbyl Phosphate | 66170-10-3 | ≤1 | Synthetic | Antioxidant |
| Xanthan Gum | 11138-66-2 | ≤1 | Biofermentation | Emulsion Stabilizer, Gel |
| | | | | former, Viscosity |
| | | | | Controller |
| Maltodextrin | 9050-36-6 | ≤1 | Vegetable | Absorbent, Binder, |
| | | | | Emulsion Stabilizer, Film |
| | | | | Former |
| Ethylhexylglycerin | 70445-33-9 | ≤1 | Synthetic | Skin Conditioner |
| Caprylyl Glycol | 1117-86-8 | ≤1 | Synthetic | Skin Conditioner, |
| | | | | Emollient |
| Hexylene Glycol | 107-41-5 | ≤1 | Synthetic | Skin Conditioner, |
| | | | | Surfactant, Emulsifier |
| Phenoxyethanol | 122-99-6 | ≤1 | Synthetic | Preservative |
| Sodium Benzoate | 532-32-1 | ≤1 | Synthetic | Preservative |
| Potassium Sorbate | 24634-61-5 | ≤1 | Synthetic | Preservative |
| Citric Acid | 77-92-9/ | ≤1 | Synthetic | Buffer |
| | 5949-29-1 | | | |
| Phytosphingosine | 13552-11-9 | ≤1 | Vegetable | Skin Conditioner |
| Cholesterol | 80356-14-5 | ≤1 | Semi- | Skin Conditioner, |
| | | | Synthetic | Emollient |
| Sodium lauroyl lactylate | 13557-75-0 | ≤1 | Natural | Surfactant, Emulsifier |
| Carbomer | 600-07-7 | ≤1 | Synthetic | Emulsion Stabilizer, Gel |
| | | | | former, Viscosity |
| | | | | Controller |

May Contain+/−

| | | | | |
|---|---|---|---|---|
| Iron oxides (CI 77492) | 51274-00-1 | ≤1 | Mineral, | Colorant |
| | | | Synthetic | |
| Iron oxides (CI 77491) | 1309-37-1 | ≤1 | Mineral, | Colorant |
| | | | Synthetic | |
| Iron oxides (CI 77499) | 1317-61-9 | ≤1 | Mineral, | Colorant |
| | | | Synthetic | |

### Example 2: Sun Protectant Testing—Static SPF Testing

### Summary

The composition prepared in Example 1 was tested under FSTI (Florida Suncare Testing, Inc.) SOP #2021-01, Static SPF Testing, and SOP #2021-02 Water Resistant SPF testing as set forth by the FDA, Over the counter monograph M020:Sunscreen Drug Products for Over-the-counter Human use (posted Sep. 24, 2021) as part of the Final Administrative order (OTC000006) effective upon enactment of the Coronavirus Aid, Relief, and Economic Security Act (CARES Act), Public Law 116-136 on Mar. 27, 2020. Part D—Testing Procedure M020.80 Sun Protection Factor (SPF) test procedure.

Table 3, below, provides a summary of the protocols followed for determining SPF of the compositions of Example 1.

TABLE 3

| | |
|---|---|
| FSTI Study number | 24-1686 |
| Study Type | FDA 2021 Static SPF |
| Method | FDA 2021 M020.80 |
| Number of subjects | 3-10 |
| Product name | Vitamin C Sunscreen Serum SPF 30 |
| Formula # | 230228-13/385-065 |
| Target SPF | 30 |

### I. Objective

The objective of the tests described in this Example was to measure the Sun Protection Factor (SPF) value for the sponsor sunscreen formula (disclosed in Example 1). Procedures described below are from Over the counter monograph M020: Sunscreen Drug Products for Over-the-counter Human use (posted Sep. 24, 2021) as party of the Final Administrative order (OTC000006) effective upon enactment of the Coronavirus Aid, Relief, and Economic Security Act (CARES Act), Public Law 116-136 on Mar. 27, 2020. Part D—Testing Procedure M020.80 Sun Protection Factor (SPF) test procedure.

### II. Experimental Design

### A. Selection of Test Subjects

Fair-skinned subjects, male and female, of legal age (18) with Skin Type Characteristics I, II, III, as defined in Over the counter monograph M020: Part D—Testing Procedure, M020.80 Sun Protection Factor Test procedure, (c) test subjects, (2) Medical history (i) (1), (2), (3), were enrolled.

The criteria used for inclusion of subject participants were: (1) Individuals, male and female, eighteen years of age or older, (2) Individuals with fair, uniformly-colored skin on the lower area of the back which would allow a discernable erythema, (3) Individuals free of any dermatological or systemic disorder which, in the opinion of the testing personnel, would interfere with the results of the study, (4) Individuals in good health who have completed a preliminary medical history, and (5) Individuals who have read, understood and signed a consent document in compliance with 21 CFR 50.

The criteria used for exclusion of subject participants were: (1) Individuals with any visible skin disease at the study site, which in the opinion of the investigative personnel would, interfere with the study results, (2) Individuals taking medications which might affect study results, e.g. photosensitizers, antihistamines, analgesics or anti-inflammatory drugs, (3) Females who are pregnant, planning a pregnancy or nursing a child, (4) Individuals with a history of skin cancer, (5) Individuals with a history of hepatitis or other blood disease, (6) Individuals with a known sensitivity to cosmetics, skin care products or topical drugs as related to product(s) being evaluated, and (7) Individuals with recent sun exposure on the areas to be tested.

Table 4 below provides a summary of the demographics of the eleven subjects enrolled in the study.

TABLE 4

| Number of Subjects Enrolled | | | | 11 | | |
|---|---|---|---|---|---|---|
| Number of Subjects completing study | | | | 11 | | |
| Age Range | | | | 28-77 | | |
| Male | 4 | | | Female | 7 | |
| Skin Types | I (0) | II (10) | III (1) | Technical failures (TF) | Static (1) | Water resistant (0) |

### B. Test Materials

A sufficient quantity (approximately 2 oz.) of each test product was submitted by the test sponsor to satisfy the requirements for a test panel of up to ten subjects. The test sample was entered into a SPF testing log and assigned a unique test product sample identification number.

### C. Light Source

A Xenon Arc Solar Simulator lamp, which provides a continuous emission spectrum from 290 to 400 nanometers (nm) with a limit of 1,500 watts per square meter (W/m2) of total irradiance for all wavelengths between 250 and 1,400 nm. The spectral output of the solar simulator will be filtered so that it meets the spectral output requirements for testing Sunscreen Drug Products for over-the-counter human use; M020.80 Sun Protection Factor (SPF) test Procedure (a) UV Source (solar simulator).

### III. Study Procedure

### A. Day 1

The test subjects reported to the testing laboratory and received a complete explanation of the study procedures. Those electing to participate signed a written consent form. The technician performed a final examination of the subject's back, between the belt line and shoulder blades to determine their suitability to participate in the study.

### MEDu UV Dose Administration

A series of 5 UV radiation doses expressed as Joules/square meter, increasing in 25% increments, were administered to an unprotected location on the test subject's back, just below the shoulder blades and above the belt-line, to determine the initial unprotected MED (MEDu).

The test subjects were instructed to avoid additional UV exposure, and to avoid taking any photosensitizing medications until the conclusion of the study.

The MEDu was administered in the following 5 dose series (Table 5), with X representing the amount of UV energy projected to produce the test subject's MEDu.

TABLE 5

| Dose 1 | Dose 2 | Dose 3 | Dose 4 | Dose 5 |
|---|---|---|---|---|
| 0.64X | 0.80X | 1.00X | 1.25X | 1.56X |

### B. Day 2

### MEDu Determination

Subjects returned to the testing laboratory within 16 to 24 hours following completion of the MEDu doses for evaluation of the responses and to determine each subject's unprotected MED (MEDu).

The subject's Minimal Erythemal Dose (MED) is the erythema effective energy, or dose corresponding to the first site that produced the first unambiguous erythema reaction with clearly defined borders. Table 6 below shows the grading scale to be used for determining a MED (+) response.

TABLE 6

| | |
|---|---|
| − | No perceptible erythemal response |
| ? | Barely perceptible erythemal response |
| + | Unambiguous erythema reaction with clearly defined borders |
| | (MED) |
| ++ | Moderate erythema with sharp borders |
| +++ | Dark red erythema with sharp borders |

### Application of Product for SPF Determination

Sunscreen application was performed as specified in: Over the counter monograph M020: Part D—Testing Procedure, M020.80 Sun Protection Factor Test procedure, (d) Sunscreen application, (3) applying test materials. Specifically, the product of Example 1 was applied to 30-60 square centimeter randomly located test sites, drawn in the designated locations on the subject's back, (between the beltline and the shoulder blade) using a template and an indelible marker. Test subsites will be a minimum of 0.5 cm2 and the distance between subsites a minimum of 0.8 cm. The technician will then apply the test formulas in the test areas. The sunscreens (product of Example 1) were applied by “spotting” the product across the test area and gently spreading, using a non-saturated finger cot compatible with the test product, until a uniform film was applied to the entire test area. A product density of 2 mg/cm2 must be delivered to the test area. To accomplish this, the technician will weigh an amount in excess to allow for the residual amount left on the finger cot (approximately 10%). The test products were permitted to dry a minimum of 15 minutes prior to UV exposure or water resistance testing.

### MEDp UV Dose Administration (Static)

The technician administered a series of 5 UV radiation doses expressed as Joules/square meter, progressively increasing in increments of either 15 percent, 20 percent, or 25 percent based on the product's target SPF as specified in OTC Monograph M020 (e) UV Exposure. The MEDp was administered in the following 5 dose series with X representing the expected amount of UV energy required to produce a MEDp based on the target SPF as listed below (Tables 7 and 8).

TABLE 7
Expected SPF 16.3 (FDA Control Static)

| Dose 1 | Dose 2 | Dose 3 | Dose 4 | Dose 5 |
|---|---|---|---|---|
| 0.76X | 0.87X | 1.00X | 1.15X | 1.32X |

TABLE 8
Expected SPF (Test Product Static and/or 40 or 80 Min WR)

| | | | | |
|---|---|---|---|---|
| Dose 1 | Dose 2 | Dose 3 | Dose 4 | Dose 5 |

Expected SPF < 8

| | | | | |
|---|---|---|---|---|
| 0.64X | 0.80X | 1.00X | 1.25X | 1.56X |

Expected SPF 8-15

| | | | | |
|---|---|---|---|---|
| 0.69X | 0.83X | 1.00X | 1.20X | 1.44X |

Expected SPF > 15

| | | | | |
|---|---|---|---|---|
| 0.76X | 0.87X | 1.00X | 1.15X | 1.32X |

### C. Day 3

### Evaluation of Responses to UV Doses for Static SPF and Repeat MEDu

Subjects will return 16 to 24 hours following completion of the UV doses from day 2. The MED for all sites that received UV doses, both protected and unprotected areas were evaluated and recorded. Erythema responses were evaluated on an area that provided at least 450 lux of illumination at the test site.

### Mean and Label SPF Computation

SPF values were calculated for all test products by calculating the ratio of the MEDp value produced in the sunscreen protected sites to the MEDu produced in the unprotected test area, for each individual using the following calculation:

$$M E D p / M E D u = S P F Value$$

Data from Ten (10) subjects was used for calculating each test product's label SPF value. The mean SPF value (x) and the Standard Deviation (s) for these subjects is computed. The upper 5-percent point from the student distribution table (denoted by t) with n−1 degrees of freedom is obtained. (1.83 based on a 10 subject panel). The quantity A is computed using the formula A=ts/Square root n (with n representing the number of test subjects included in this study). The label SPF is calculated by determining the largest whole number less than X−A. Any test product with a label SPF less than 2 is not a sunscreen drug product and will not display an SPF value.

### IV. Results

### Test Composition Results

Table 9, below, provides a summary of the results of the Static SPF testing of the composition from Example 1. Note that water resistance (WR) was not tested.

TABLE 9

| | | | |
|---|---|---|---|
| FSTI Study | 24-1686 | Sponsor Formula | 230228-13/ |
| Number | | Number | 385-065 |
| Mean Static | Label Static | Mean WR SPF | Label WR SPF |
| SPF | SPF | | |
| 33.86 | 31 | N/A | N/A |

### Reference Sunscreen Standard

The results of the FDA standard tested as part of this study had a mean static SPF value within the specified range of 16.3+3.43 and satisfied all statistical criteria as stated in M020.80 Sun Protection Factor (SPF) test procedure (f) Determination of SPF. The results of the P2 standard can be seen in the table below.

| | |
|---|---|
| FSTI Study Number | 24-1686 |
| P2 Mean Static SPF Value | 17.53 |

Tables 10A and 10B. below, provides a summary of all sponsor test sample data yielded in the study.

TABLE 10A

| | | | | | | | Skin | |
|---|---|---|---|---|---|---|---|---|
| Subject | App | Exp | Read | Subject | Sex | | Type | MEDu |
| No | by | by | by | ID No. | M/F | Age | I/II/III | J/m² |
| 1 | AM | AM | MR | 3049 | F | 66 | II | 79.33 |
| 2 | AM | AM | MR | 7107 | M | 42 | II | 79.33 |
| 3 | AM | DK | MR | 7005 | M | 77 | III | 99.17 |
| 4 | AM | DK | MR | 7142 | M | 56 | II | 79.33 |
| 5 | AM | AM | MR | 7126 | F | 33 | II | 79.33 |
| 6 | AM | AM | MR | 7143 | F | 28 | II | 79.33 |
| 7 | AM | AM | MR | 7121 | M | 73 | II | 79.33 |
| 8 | AM | AM | MR | 3357 | F | 64 | II | 79.33 |
| 9 | AM | AM | MR | 7132 | F | 57 | II | 79.33 |
| 10 | AM | AM | MR | 3433 | F | 55 | II | 79.33 |
| 11 | AM | AM | MR | 7105 | F | 76 | II | 79.33 |

### V. Conclusions

TABLE 10B

| | MEDp J/m² | | | SPF | | |
|---|---|---|---|---|---|---|
| | 1902AE | 24-1686 | 24-1686 | 1902AE | 24-1686 | |
| Subject | FDA Std. | Static | WR | FDA Std. | Static | 24-1686 |
| No | J/m² | J/m² | J/m² | Static SPF | SPF | WR |
| 1 | 1293 | 2069 | N/A | 16.3 | 26.09 | N/A |
| 2 | 1293 | 2380 | N/A | 16.3 | 30 | N/A |
| 3 | 1859 | 3935 | N/A | 18.75 | 39.68 | N/A |
| 4 | 1487 | 3147 | N/A | 18.75 | 39.68 | N/A |
| 5 | 1293 | 1800 | N/A | 16.30 * | 22.86 TF | N/A |
| 6 | 1293 | 2380 | N/A | 16.3 | 30 | N/A |
| 7 | 1487 | 2737 | N/A | 18.75 | 34.5 | N/A |
| 8 | 1487 | 3147 | N/A | 18.75 | 39.68 | N/A |
| 9 | 1293 | 2380 | N/A | 16.3 | 30 | N/A |
| 10 | 1487 | 2737 | N/A | 18.75 | 34.5 | N/A |
| 11 | 1293 | 2737 | N/A | 16.3 | 34.5 | N/A |

The results of the experiments described herein confirmed that the product described in Example 1 had a mean Sun Protection Factor (SPF) of 33.86 and a label static SPF factor of 31.

### Example 3: Repeated Insult Patch Test

### I. Objective

The composition prepared in Example 1 was tested using a Repeated Insult Patch test to confirm, in healthy human subjects, that the test product was not associated with a dermal sensitization (allergic) response under repeated occlusive patch test conditions.

### II. Test Subjects

At least 50 male and female subjects ranging in age from 18 to 79 years were enrolled in the study. Subject demographics are listed in Table A below. The subjects did not exhibit any physical or dermatological conditions that would have precluded application of the test article or determination of potential effects of the test article.

### III. Test Procedure

The 9 Repeated Insult (occlusive) Patch Test (9-RIPT) was performed as follows and as described in Marzuli F N et al., ((1976) “Contact Allergy: Predictive testing in man” Contact Dermatitis. 2, 1-17. Essex Testing Clinic, Inc, incorporated herein by reference in its entirety).

### Induction Phase

A sufficient amount of the test article (approximately 0.1 g-0.15 g) was placed onto a Parke-Davis Readi-Bandage® occlusive patch (approximately 25-38 mg/cm² of test material) and applied to the back of each subject between the scapulae and waist, adjacent to the spinal mid-line. This procedure was performed by a trained technician/examiner and repeated every Monday, Wednesday and Friday until 9 applications of the test article had been made.

The subjects were instructed to remove the patch 24 hours after application. Twenty-four hour rest periods followed the Tuesday and Thursday removals and 48-hour rest periods followed each Saturday removal. Subjects returned to the Testing Facility and the site was scored by a trained examiner just prior to the next patch application.

If a subject developed a positive reaction of a level 2 erythema or greater during the Induction phase or if, at the discretion of the Study Director, the skin response warranted a change in site, the patch was applied to a previously unpatched, adjacent site for the next application. If a level 2 reaction or greater occurred at the new site, no further applications were made. However, any reactive subjects were subsequently Challenge patch tested.

### Challenge Phase

After a rest period of approximately 2 weeks (no applications of the test article), the Challenge patch was applied to a previously unpatched (virgin) test site. The subjects returned to the Testing Facility 24 hours after patch application and the patch was removed. The site was scored immediately after patch removal. Subjects returned to the Testing Facility 72 hours after application (48 hours after removal) and the site was scored again. All subjects were instructed to report any delayed skin reactivity that occurred after the final Challenge patch reading. When warranted, selected test subjects were called back to the Clinic for additional examinations and scoring to determine possible increases or decreases in Challenge patch reactivity.

### Scoring of Dermal Responses

Dermal responses for both the Induction and Challenge phases of the study were scored according to the following 6-point scale.
0=No evidence of any effect
+=Barely perceptible (Minimal, faint, uniform or spotty erythema)
1=Mild (Pink, uniform erythema covering most of the contact site)
2=Moderate (Pink-red erythema uniform in the entire contact site)
3=Marked (Bright red erythema with/without petechiae or papules)
4=Severe (Deep red erythema with/without vesiculation or weeping)

All other observed dermal sequelae (e.g., edema, dryness, hypo- or hyperpigmentation) were appropriately recorded on the data sheet and described as mild, moderate, or severe.

### Data Interpretation

Edema, vesicles, papules, and/or erythema that persist or increase in intensity either during the Induction and/or Challenge phase may be indicative of allergic contact dermatitis. Allergic responses normally do not resolve or improve markedly at 72-96 hours.

Exceptions to typical skin reactions may occur. These may include, but not be limited to, symptoms of allergic contact sensitivity early in the Induction period to one or more test products. When this occurs in one subject, such a reaction usually suggests either an idiosyncratic response or that the subject had a preexposure/sensitization to the test material or component(s) of the test material or a cross-reactivity with a similar product/component. Data for such reactions will be included in the study report but will not be included in the final study analysis/conclusion of sensitization.

### IV. Results and Discussion

A total of 60 subjects (19 males and 41 females ranging in age from 22 to 74 years) were empaneled for the test procedure. Fifty-six (56/60) subjects satisfactorily completed the test procedure on Test Article: Vitamin C Sunscreen Serum SPF 30—230228-13/385-065. Four (4/60) subjects discontinued for personal reasons unrelated to the conduct of the study. Discontinued subject data are shown up to the point of discontinuation, but are not used in the Conclusions section of this final report.

Tables 12 and 13 provide summaries of Induction Phase and Challenge Phase results. As shown in both tables, no skin reactivity was observed at any time during the course of the study. Table B below provide a full breakdown of results from each participant.

TABLE 12
Induction Phase Summary

| | Induction Scores | | | | | | Evidence |
|---|---|---|---|---|---|---|---|
| | (Number of Responses) | | | | | | of |
| Test Article | 0.5 | 1 | 2 | 3 | 4 | Other | Irritation |
| Vitamin C Sunscreen Serum | 0 | 0 | 0 | 0 | 0 | 0 | No |
| SPF 30 - 230228-13/385-065 | | | | | | | |

TABLE 13
Challenge Phase Summary

| | Challenge Scores | | | | | | Evidence |
|---|---|---|---|---|---|---|---|
| | (Number of Responses) | | | | | | of |
| Test Article | 0.5 | 1 | 2 | 3 | 4 | Other | Irritation |
| Vitamin C Sunscreen Serum | 0 | 0 | 0 | 0 | 0 | 0 | No |
| SPF 30 - 230228-13/385-065 | | | | | | | |

### V. Conclusions

Under the conditions of a repeated insult (occlusive) path test procedure conducted in 56 subjects, Test Article: Vitamin C Sunscreen Serum SPF 30—230228-13/385-065 was “Dermatologist-Tested” and was not associated with skin irritation or allergic contact dermatitis in human subjects.

TABLE A
Subject Demographics

| Subject No. | Initials | Age | Sex | Race | Fitzpatrick Skin Type | Skin Type | Sensitive Skin |
|---|---|---|---|---|---|---|---|
| 1 | G-M | 55 | F | OT | IV | Normal | Yes |
| 2 | N•B | 22 | M | WH | II | Normal | Yes |
| 3 | M-W | 56 | F | BA | IV | Normal/Oily | Yes |
| 4 | A•M | 56 | F | AS | IV | Oily/Normal | No |
| 5 | R-K | 55 | M | WH | I | Oily | Yes |
| 6 | J•N | 68 | M | AS | IV | Normal | Yes |
| 7 | R•B | 70 | M | BA | III | Normal | No |
| 8 | J-J | 54 | F | WH | II | Normal | Yes |
| 9 | L•P | 44 | F | WH | III | Oily/Normal | Yes |
| 10 | P•A | 55 | F | BA | VI | Normal | No |
| 11 | 0-R | 57 | F | BH | IV | Normal | Yes |
| 12 | B•H | 70 | F | BA | VI | Normal/Oily | No |
| 13 | T-0 | 64 | F | WH | IV | Normal | No |
| 14 | K•M | 67 | F | WH | II | Normal/Oily | Yes |
| 15 | A•C | 63 | F | WH | IV | Normal | Yes |
| 16 | R•E | 45 | F | HS | IV | Normal | No |
| 17 | R-M | 68 | F | WH | III | Normal | Yes |
| 18 | M•L | 45 | M | BA | VI | Normal | No |
| 19 | O•L | 42 | F | WH | I | Normal | Yes |
| 20 | Y-K | 74 | F | WH | I | Oily/Oily | No |
| 21 | T•S | 29 | F | BA | VI | Normal/Oily | Nc |
| 22 | K•C | 67 | M | WH | III | Oily/Normal | Yes |
| 23 | K-C | 67 | F | WH | II | Dry/Normal | Yes |
| 24 | T-W | 25 | F | BA | VI | Normal | No |
| 25 | W-T | 67 | F | OT | III | Normal | Yes |
| 26 | D-J | 72 | F | BA | III | Normal/Oily | No |
| 27 | N-J | 43 | F | BA | VI | Oily/Normal | No |
| 28 | M-F | 65 | M | WH | III | Normal/Oily | Yes |
| 29 | T-B | 59 | F | BH | V | Normal | No |
| 30 | G-G | 67 | M | WH | IV | Normal | Yes |
| 31 | L•G | 61 | F | WH | IV | Normal | Yes |
| 32 | R-D | 59 | M | WH | III | Normal | Yes |
| 33 | M•P | 54 | M | WH | III | Normal | No |
| 34 | L-M | 73 | F | WH | IV | Oily/Normal | Yes |
| 35 | J-L | 30 | F | HS | IV | Normal | No |
| 36 | N-H | 74 | F | BH | V | Normal/Oily | No |
| 37 | Y•L | 55 | F | BA | V | Normal | Yes |
| 38 | N-K | 67 | F | WH | III | Normal | Yes |
| 39 | D-R | 64 | M | BA | VI | Normal | No |
| 40 | R-M | 65 | F | WH | III | Oily/Normal | Yes |
| 41 | M•A | 62 | F | WH | IV | Normal | Yes |
| 42 | T•E | 65 | M | BA | VI | Normal/Oily | No |
| 43 | A•I | 31 | F | BH | IV | Normal | No |
| 44 | I-N | 52 | F | HS | IV | Normal | No |
| 45 | P-M | 71 | F | WH | III | Normal/Oily | No |
| 46 | L•M | 40 | M | BA | VI | Normal/Oily | No |
| 47 | T-B | 22 | M | BA | V | Normal/Oily | No |
| 48 | S•M | 40 | M | BA | VI | Normal | No |
| 49 | A•S | 64 | F | BA | VI | Normal/Oily | No |
| 50 | M-T | 63 | F | WH | III | Normal | Yes |
| 51 | M-H | 55 | F | WH | III | Normal/Oily | Yes |
| 52 | I•W | 36 | M | BA | VI | Normal | Nc |
| 53 | K•H | 26 | F | WH | II | Oily/Oily | Yes |
| 54 | T•B | 70 | F | BA | VI | Normal | No |
| 55 | A•V | 55 | M | HS | IV | Normal | No |
| 56 | S•P | 57 | F | OT | III | Normal | No |
| 57 | G•C | 61 | F | BA | V | Normal | Yes |
| 58 | M-Z | 69 | F | WH | II | Normal | Yes |
| 59 | Y-S | 61 | M | WH | III | Normal | Yes |
| 60 | R-J | 52 | M | WH | III | Normal | Yes |

Legend for Table A:
Based on Fitzpatrick Scale
Skin Type I • Always burns easily; never tans (sensitive)
Skin Type II • Always burns easily; tans minimally (sensitive)
Skin Type III • Burns moderately; tans gradually (light brown/normal)
Skin Type IV - Burns minimally; always tans well (moderate brown/normal)
Skin Type V • Rarely burns; tans profusely (dark brown/insensitive)
Skin Type VI • Never burns; deeply pigmented (insensitive)
Race
AS =Asian
BA = Black
BH = Black/Hispanic
HS = Hispanic/Latino
WH = White
OT = Other

TABLE B
Individual Scores - Repeated Insult Patch Test - Occlusive

| | | | | | | | | | | Challenge | |
|---|---|---|---|---|---|---|---|---|---|---|---|
| Subject | Induction Evaluation Number | | | | | | | | | Virgin Site | |
| No. | 1 | 2 | 3 | 4 | 5 | 6 | 7 | 8 | 9 | 24 hr | 72 hr |
| 1 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 |
| 6 | 0 | 0 | 0 | 0 | 0 | 0 | 0 | 0 | 0 | 0 | 0 |
| 7 | 0 | 0 | 0 | 0 | 0 | 0 | 0 | 0 | 0 | 0 | 0 |
| 8 | 0 | 0 | 0 | 0 | 0 | 0 | 0 | 0 | 0 | 0 | 0 |
| 9 | 0 | 0 | 0 | 0 | 0 | 0 | 0 | 0 | 0 | 0 | 0 |
| 10 | 0 | 0 | 0 | 0 | 0 | 0 | 0 | 0 | 0 | 0 | 0 |
| 11 | 0 | 0 | 0 | 0 | 0 | 0 | 0 | 0 | 0 | 0 | 0 |
| 12 | 0 | 0 | 0 | 0 | 0 | 0 | 0 | 0 | 0 | 0 | 0 |
| 13 | Discontinued | | | | | | | | | | |
| 14 | 0 | 0 | 0 | 0 | 0 | 0 | 0 | 0 | 0 | 0 | 0 |
| 15 | 0 | 0 | 0 | 0 | 0 | 0 | 0 | 0 | 0 | 0 | 0 |
| 16 | 0 | 0 | 0 | 0 | 0 | 0 | 0 | 0 | 0 | 0 | 0 |
| 17 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0 |
| 19 | 0 | 0 | 0 | 0 | 0 | 0 | 0 | 0 | 0 | 0 | 0 |
| 20 | 0 | 0 | 0 | 0 | 0 | 0 | 0 | 0 | 0 | 0 | 0 |
| 21 | 0 | 0 | 0 | 0 | 0 | 0 | 0 | 0 | 0 | Discontinued | |
| 22 | 0 | 0 | 0 | 0 | 0 | 0 | 0 | 0 | 0 | 0 | 0 |
| 23 | 0 | 0 | 0 | 0 | 0 | 0 | 0 | 0 | 0 | 0 | 0 |
| 24 | 0 | 0 | 0 | Discontinued | | | | | | | |
| 25 | 0 | 0 | 0 | 0 | 0 | 0 | 0 | 0 | 0 | 0 | 0 |
| 26 | 0 | 0 | 0 | 0 | 0 | 0 | 0 | 0 | 0 | 0 | 0 |
| 27 | 0 | 0 | 0 | 0 | 0 | 0 | 0 | 0 | 0 | 0 | 0 |
| 28 | 0 | 0 | 0 | 0 | 0 | 0 | 0 | 0 | 0 | 0 | 0 |
| 29 | 0 | 0 | 0 | 0 | 0 | 0 | 0 | 0 | 0 | 0 | 0 |
| 30 | 0 | 0 | 0 | 0 | 0 | 0 | 0 | 0 | 0 | 0 | 0 |
| 31 | 0 | 0 | 0 | 0 | 0 | 0 | 0 | 0 | 0 | 0 | 0 |
| 32 | 0 | 0 | 0 | 0 | 0 | 0 | 0 | 0 | 0 | 0 | 0 |
| 33 | 0 | 0 | 0 | 0 | 0 | 0 | 0 | 0 | 0 | 0 | 0 |
| 34 | 0 | 0 | 0 | 0 | 0 | 0 | 0 | 0 | 0 | 0 | 0 |
| 35 | 0 | 0 | 0 | 0 | 0 | 0 | 0 | 0 | 0 | 0 | 0 |
| 36 | 0 | 0 | 0 | 0 | 0 | 0 | 0 | 0 | 0 | 0 | 0 |
| 37 | 0 | 0 | 0 | 0 | 0 | 0 | 0 | 0 | 0 | 0 | 0 |
| 38 | 0 | 0 | 0 | 0 | 0 | 0 | 0 | 0 | 0 | 0 | 0 |
| 39 | 0 | 0 | 0 | 0 | 0 | 0 | 0 | 0 | 0 | 0 | 0 |
| 40 | 0 | 0 | 0 | 0 | 0 | 0 | 0 | 0 | 0 | 0 | 0 |
| 41 | 0 | 0 | 0 | 0 | 0 | 0 | 0 | 0 | 0 | 0 | 0 |
| 42 | 0 | 0 | 0 | 0 | 0 | 0 | 0 | 0 | 0 | 0 | 0 |
| 43 | 0 | 0 | 0 | 0 | 0 | 0 | 0 | 0 | 0 | 0 | 0 |
| 44 | 0 | 0 | 0 | 0 | 0 | 0 | 0 | 0 | 0 | 0 | 0 |
| 45 | 0 | 0 | 0 | 0 | 0 | 0 | 0 | 0 | 0 | 0 | 0 |
| 46 | 0 | 0 | 0 | 0 | 0 | 0 | 0 | 0 | 0 | 0 | 0 |
| 47 | 0 | 0 | 0 | 0 | 0 | 0 | 0 | 0 | 0 | 0 | 0 |
| 48 | 0 | 0 | 0 | 0 | 0 | 0 | 0 | 0 | 0 | 0 | 0 |
| 49 | 0 | 0 | 0 | 0 | 0 | 0 | 0 | 0 | 0 | 0 | 0 |
| 50 | 0 | 0 | 0 | 0 | 0 | 0 | 0 | 0 | 0 | 0 | 0 |
| 51 | 0 | 0 | 0 | 0 | 0 | 0 | 0 | 0 | 0 | 0 | 0 |
| 52 | 0 | 0 | 0 | 0 | 0 | 0 | 0 | 0 | 0 | Discontinued | |
| 53 | 0 | 0 | 0 | 0 | 0 | 0 | 0 | 0 | 0 | 0 | 0 |
| 54 | 0 | 0 | 0 | 0 | 0 | 0 | 0 | 0 | 0 | 0 | 0 |
| 55 | 0 | 0 | 0 | 0 | 0 | 0 | 0 | 0 | 0 | 0 | 0 |
| 56 | 0 | 0 | 0 | 0 | 0 | 0 | 0 | 0 | 0 | 0 | 0 |
| 57 | 0 | 0 | 0 | 0 | 0 | 0 | 0 | 0 | 0 | 0 | 0 |
| 58 | 0 | 0 | 0 | 0 | 0 | 0 | 0 | 0 | 0 | 0 | 0 |
| 59 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 |

Legend for Table B
0 = No evidence of any effect
+ = Barely perceptible (Minimal, faint, uniform or spotty erythema)
1 = Mild (Pink, uniform erythema covering most of the contact site)
2 = Moderate (Pink-red erythema uniform in the entire contact site)
3 = Marked (Bright red erythema with/without petechiae or papules)
4 = Severe (Deep red erythema with/without vesiculation or weeping)

### Example 4: Antimicrobial Effectiveness Testing

An antimicrobial effectiveness test was conducted on the Vitamin C Sunscreen Serum SPF 30 in accordance with United States Pharmacopeia (USP) <51> Category 2 guidelines. The test evaluates the preservative system's ability to inhibit microbial growth over a 28-day period. Samples were inoculated with specified challenge microorganisms and incubated at 20-25° C. Microbial counts were taken initially, at 14 days, and at 28 days to determine log reductions and assess compliance with USP acceptance criteria.

The microorganisms tested included Escherichia coli (ATCC 8739), Pseudomonas aeruginosa (ATCC 9027), Staphylococcus aureus (ATCC 6538), Candida albicans (ATCC 10231), and Aspergillus brasiliensis (ATCC 16404). For bacterial strains, USP <51> requires at least a 2.0-log reduction at 14 days with no increase at 28 days. For yeast and mold, no increase is permitted at 14 or 28 days. Based on the reported microbial counts and log reductions, the product met all USP <51> Category 2 criteria and was deemed compliant.

| | Initial | 14 Days | 14 Days | 28 Days | 28 Days |
|---|---|---|---|---|---|
| | Count | Count | Log Re- | Count | Log Re- |
| Microorganism | (cfu) | (cfu) | duction | (cfu) | duction |
| E. coli (ATCC | 4.1 × 10\hat{ }5 | 0 × 10\hat{ }1 | 4.61 | 0 × 10\hat{ }1 | 4.61 |
| 8739) | | | | | |
| P. aeruginosa | 6.7 × 10\hat{ }5 | 0 × 10\hat{ }1 | 4.83 | 0 × 10\hat{ }1 | 4.83 |
| (ATCC 9027) | | | | | |
| S. aureus | 7.9 × 10\hat{ }5 | 0 × 10\hat{ }1 | 4.90 | 0 × 10\hat{ }1 | 4.90 |
| (ATCC 6538) | | | | | |
| C. albicans | 5.7 × 10\hat{ }5 | 0 × 10\hat{ }1 | 4.76 | 0 × 10\hat{ }1 | 4.76 |
| (ATCC 10231) | | | | | |
| A. brasiliensis | 3.8 × 10\hat{ }5 | 1.3 × 10\hat{ }2 | 3.47 | 0 × 10\hat{ }1 | 4.58 |
| (ATCC 16404) | | | | | |

### Example 5: Microbial Limit Test (Method Suitability)

A method suitability assessment for Microbial Limit Testing (MLT) was performed on the Vitamin C Sunscreen Serum SPF 30 in accordance with USP <61>, <62>, and <1227>. The objective was to demonstrate that the product matrix, when appropriately diluted, does not inhibit recovery of viable microorganisms and that any antimicrobial preservative activity can be neutralized to permit accurate enumeration. The evaluation compared recoveries of challenge organisms from product test preparations against positive controls prepared without product.

Test preparations employed Modified Letheen Broth (MLB) as the diluent at a 1:10 dilution for both aerobic plate count (APC) and yeast and mold testing. Recovery percentages for all USP-specified challenge organisms fell within the acceptance range of 50% to 200% relative to positive controls, thereby validating the method for the subject formulation.

| Microorganism (ATCC) | Recovery (%) | Remark |
|---|---|---|
| E. coli (ATCC 8739) | 119 | Pass |
| S. aureus (ATCC 6538) | 113 | Pass |
| P. aeruginosa (ATCC 9027) | 94 | Pass |
| B. spizizenii (ATCC 6633) | 109 | Pass |
| C. albicans (ATCC 10231) | 89 | Pass |
| A. brasiliensis (ATCC 16404) | 106 | Pass |

Based on these results, the suggested diluent & dilution for APC was MLB, 1:10 and the suggested diluent & dilution for Yeast & Mol was MLB, 1:10.

### Example 6: Accelerated Stability & Freeze-Thaw Study

A six-month accelerated stability evaluation was conducted on the Vitamin C Sunscreen SPF 30 (Product Code 230228-13/385-065; Lot 230228-13-05) to assess physical, chemical, microbiological, and packaging compatibility attributes under accelerated conditions. Testing followed an internal protocol referenced as Report No. A/Stability Report/2025/28. Samples were packaged in a 1 oz amber bottle with a black pump and incubated beginning 10/17/2024, with completion dated Aug. 13, 2025.

Acceptance specifications included appearance, color, odor, viscosity, zinc oxide assay (target 14.5-17.7%), and microbial limits (APC ≤100 cfu/g; yeast & mold ≤10 cfu/g; absence per gram of E. coli, S. aureus, and P. aeruginosa). Packaging compatibility was monitored for decoloration, leakage, delamination, and unit weight loss over time. All results remained within specification throughout the 6-month study.

Declared Specifications

| Category | Parameter | Specification |
|---|---|---|
| Physical | Appearance | Low Viscosity Serum |
| Physical | Color | Beige |
| Physical | Odor | Characteristic |
| Physical | Viscosity (LVT4, 6 RPM) | Approx. 2,000 cP |
| Chemical | Zinc Oxide | 14.5-17.7% |
| Microbiological | Aerobic Plate Count | Not more than 100 cfu/g |
| Microbiological | Yeast & Mold | Not more than 10 cfu/g |
| Microbiological | Pathogens | E. coli, S. aureus, P. |
| | | aeruginosa: Absent/g |

Accelerated Stability Results (Room Temp & 40° C./75% RH)

| | | | 1st Month | | 3rd Month | | 6th Month | | Within |
|---|---|---|---|---|---|---|---|---|---|
| Test | Initial (RT) | | (40° C./75% RH) | | (40° C./75% RH) | | (40° C./75% RH) | | Spec? |
| Appearance | Low Viscosity | | Low Viscosity | | Low Viscosity | | Low Viscosity | | Yes |
| | Serum | | Serum | | Serum | | Serum | | |
| Color | Beige | | Beige | | Beige | | Beige | | Yes |
| Odor | Characteristic | | Characteristic | | Characteristic | | Characteristic | | Yes |
| Viscosity | 2,000 | cP | 2,000 | cP | 10,000 | cP | 12,000 | cP | Yes |
| (LVT4, 6 | | | | | | | | | |
| RPM) | | | | | | | | | |
| Zinc Oxide | 16.50% | | 16.40% | | 16.30% | | 16.20% | | Yes |
| APC | <10 | cfu/g | <10 | cfu/g | <10 | cfu/g | <10 | cfu/g | Yes |
| Yeast & | <10 | cfu/g | <10 | cfu/g | <10 | cfu/g | <10 | cfu/g | Yes |
| Mold | | | | | | | | | |

| | | | | | |
|---|---|---|---|---|---|
| E. coli | Absent/g | Absent/g | Absent/g | Absent/g | Yes |
| S. aureus | Absent/g | Absent/g | Absent/g | Absent/g | Yes |
| P. aeruginosa | Absent/g | Absent/g | Absent/g | Absent/g | Yes |

Packaging Compatibility - Visual Observations

| | Package | Package | | | |
|---|---|---|---|---|---|
| Timepoint | Container | Cap | Decoloration | Leakage | Delamination |
| Initial | Amber | Black Pump | None | None | None |
| | Bottle | | | Observed | Observed |
| 1st Month | No | No | None | None | None |
| | Decoloration | Decoloration | | Observed | Observed |
| 3rd Month | No | No | None | None | None |
| | Decoloration | Decoloration | | Observed | Observed |
| 6th Month | No | No | None | None | None |
| | Decoloration | Decoloration | | Observed | Observed |

Packaging Compatibility - Weight Loss

| | | 1st | | 3rd | | 6th | |
|---|---|---|---|---|---|---|---|
| | Initial | Month | % | Month | % | Month | % |
| Bottle | Weight | Weight | Loss | Weight | Loss | Weight | Loss |
| # | (g) | (g) | (1st) | (g) | (3rd) | (g) | (6th) |
| 1 | 86.8003 | 86.7023 | 0.11% | 86.5997 | 0.23% | 86.4828 | 0.37% |
| 2 | 85.2828 | 85.1746 | 0.13% | 85.0959 | 0.22% | 84.9833 | 0.35% |
| 3 | 86.2478 | 86.1329 | 0.13% | 86.0715 | 0.20% | 85.9651 | 0.33% |

### Freeze-Thaw Study (3 Cycles)

Packaging: 1 oz amber bottle with black pump. Physical targets prior to cycling: Low viscosity serum; beige; characteristic odor; viscosity approx. 2,000 cP.

| | | Temp | Temp | | | Viscosity | |
|---|---|---|---|---|---|---|---|
| | Condition | In | Out | | | (LVT4, 6 | |
| Cycle | In/Out | (° C.) | (° C.) | Appearance | Color | RPM) | Odor |
| 1 - | Oct. 21, 2024 | −16.5 | −16.9 | Low | Beige | 2,000 cP | Characteristic |
| Freezer | → | | | Viscosity | | | |
| | Oct. 22, 2024 | | | Serum | | | |
| 1 - | Oct. 22, 2024 | 46.8 | 47.0 | Low | Beige | 2,000 cP | Characteristic |
| Incubator | → | | | Viscosity | | | |
| | Oct. 23, 2024 | | | Serum | | | |
| 2 - | Oct. 28, 2024 | −16.5 | −17.3 | Low | Beige | 2,000 cP | Characteristic |
| Freezer | → | | | Viscosity | | | |
| | Oct. 29, 2024 | | | Serum | | | |
| 2 - | Oct. 29, 2024 | 46.2 | 47.1 | Low | Beige | 1,500 cP | Characteristic |
| Incubator | → | | | Viscosity | | | |
| | Oct. 30, 2024 | | | Serum | | | |
| 3 - | Nov. 4, 2024 | −16.9 | −17.3 | Low | Beige | 2,000 cP | Characteristic |
| Freezer | → | | | Viscosity | | | |
| | Nov. 5, 2024 | | | Serum | | | |
| 3 - | Nov. 5, 2024 | 47.8 | 47.9 | Low | Beige | 2,500 cP | Characteristic |
| Incubator | → | | | Viscosity | | | |
| | Nov. 6, 2024 | | | Serum | | | |

Conclusion: All tests were conducted per the referenced stability protocol. The product met specifications for physical, microbiological, and chemical attributes at all measured timepoints under accelerated conditions. Freeze-thaw cycling (three cycles) showed no separation and maintained acceptable appearance, color, odor, and viscosity.

1. A composition comprising zinc oxide, an antioxidant, and at least one plant extract.

2. The composition of claim 1, wherein the antioxidant comprises ascorbic acid or a derivative thereof, and/or the at least one plant extract is selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

3. The composition of claim 1, wherein the antioxidant comprises sodium ascorbyl phosphate (SAP).

4. The composition of claim 1, wherein the antioxidant comprises up to 1% by weight of the total composition.

5. (canceled)

6. The composition of claim 1, wherein the composition comprises at least two or at least three plant extracts.

7-8. (canceled)

9. The composition of claim 1, comprising up to 1% of each of the least one plant extracts, up to 1% of the antioxidant, and about 10% to about 30% zinc oxide.

10-14. (canceled)

15. The composition of claim 1, further comprising at least one skin conditioner, a humectant, a non-aqueous solvent, a surfactant/emulsifier/emulsion stabilizer, or any combination thereof.

16. The composition of claim 15, wherein the at least one skin conditioner comprises caprylic/capric triglyceride, coconut alkanes, isopropyl isostearate, or any combination thereof, the humectant comprises glycerin, the non-aqueous solvent comprises C13-C15 alkane, and/or the surfactant/emulsifier/emulsion stabilizer comprises Polyglyceryl-4 diisostearate/polyhydroxystearate/sebacate.

17-19. (canceled)

20. The composition of claim 15, wherein the composition comprises a first skin conditioner comprising about 15% to about 25% by weight of the total composition, a second skin conditioner comprising about 1% to 5% by weight of the total composition, and a third skin conditioner comprising about 1% to 5% by weight of the total composition; a humectant comprising about 1 to 5% by weight of the total composition; a non-aqueous solvent comprising about 1 to 5% by weight of the total composition; and a surfactant/emulsifier/emulsion stabilizer comprising about 1 to 5% by weight of the total composition.

21-33. (canceled)

34. The composition of claim 15, comprising 16.1% zinc oxide, up to 1% sodium ascorbyl phosphate (SAP), up to 1% of Camellia sinensis(green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract, 15% to about 25% caprylic/capric triglyceride, 1% to 5% isopropyl isostearate, 1% to 5% coconut alkanes, 1 to 5% glycerin, 1 to 5% of C13-C15 alkane and 1 to 5% of polyglyceryl-4 diisostearate/polyhydroxystearate/sebacate.

35. The composition of claim 1, further comprising one or more additional ingredients selected from a skin conditioner, a second antioxidant, a humectant, a nonaqueous solvent, an emollient, an emulsifier, a surfactant, a bulking agent, a viscosity controller, a cleanser, a gelling agent, a film former, a preservative, and a buffer.

36. (canceled)

37. The composition of claim 35, wherein the one or more additional ingredients are selected from sodium chloride, niacinamide, sodium hyaluronate, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, citric acid, phytosphingosine, cholesterol, sodium lauroyl lactylate, and carbomer.

38. The composition of claim 35, wherein each of the one or more additional ingredients are present at a concentration of up to 1% by weight of the total composition.

39. The composition of claim 1, further comprising one or more colorants.

40. The composition of claim 39, wherein the colorants comprise iron oxides.

41. A composition comprising zinc oxide, caprylic/capric triglyceride, isopropyl isostearate, coconut alkanes, polyglyceryl-4 diisostearate/polyhydroxystearate/sebacate, C13-15 Alkane, glycerin, sodium ascorbyl phosphate, and at least one plant extract selected from Camellia sinensis (green tea) leaf extract, Opuntia ficus-indica stem (prickly pear) extract, Caesalpinia spinosa (spiny holdback) fruit pod extract, and Helianthus annuus (sunflower) sprout extract.

42. The composition of claim 41, comprising 16.1% zinc oxide, 15 to 25% caprylic/capric triglyceride, 1 to 5% of isopropyl isostearate, 1 to 5% of coconut alkanes, 1 to 5% of polyglyceryl-4 diisostearate/polyhydroxystearate/sebacate, 1 to 5% of C13-15 Alkane, 1 to 5% of glycerin, up to 1% sodium ascorbyl phosphate, up to 1% of Camellia sinensis (green tea) leaf extract, up to 1% of Opuntia ficus-indica stem (prickly pear) extract, up to 1% of Caesalpinia spinosa (spiny holdback) fruit pod extract, and up to 1% of Helianthus annuus (sunflower) sprout extract.

43. (canceled)

44. The composition of claim 41, further comprising one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more of sodium chloride, niacinamide, sodium hyaluronate, coco-caprylate/caprate, polyhydroxystearic acid, polyglyceryl-3 polyricinoleate, isostearic acid, lecithin, ceramide NP, ceramide AP, ceramide EOP, xanthan gum, maltodextrin, ethylhexylglycerin, caprylyl glycol, hexylene glycol, phenoxyethanol, sodium benzoate, potassium sorbate, and citric acid, each at a concentration up to 1% by weight of the total composition.

45. (canceled)

46. The composition of claim 1, formulated for topical administration.

47. The composition of claim 46, wherein the composition is a topical serum, ointment, or cream.

48. (canceled)

49. A method of protecting a skin surface from sun exposure in a subject in need thereof, the method comprising topically applying a composition of claim 1 to the surface of the subject's skin.

50-56. (canceled)
